SEQUENCE LISTING (2 sequences)

SEQ ID NO: 1 - DNA, 1983 bp, Homo sapiens
  ref <308>: DATABASE ACCESSION NUMBER: U97669
  ref <309>: DATABASE ENTRY DATE: 1997-12-17
  ref <313>: RELEVANT RESIDUES IN SEQ ID NO: (5062)..(7044)
gtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcaggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggtggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctga

SEQ ID NO: 2 - protein, 660 aa, Homo sapiens
  ref <308>: DATABASE ACCESSION NUMBER: NP_000426
  ref <309>: DATABASE ENTRY DATE: 2015-03-15
  ref <313>: RELEVANT RESIDUES IN SEQ ID NO: (1662)..(2321)
VMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

(19) United States
(12) Patent Application Publication
(10) Pub. No.: US 2016/0032244 A1
(43) Pub. Date: Feb. 4, 2016

(54) Method of inducing cellular differentiation using the Notch3 receptor intracellular domain

(71) Applicant: Gabriel Rusanescu, Somerville, MA (US)

(72) Inventor: Gabriel Rusanescu, Somerville, MA (US)

(21) Appl. No.: 14/817,188

(22) Filed: Aug. 3, 2015

Related U.S. Application Data
(60) Provisional application No. 62/033,058, filed on Aug. 4, 2014.

(51) Int. Cl. C12N 5/0793; C12N 5/077

(52) U.S. Cl. CPC C12N 5/0619; C12N 5/0654; C12N 2501/42

(57) ABSTRACT

The present invention relates to the intracellular domain of Notch3 that can activate signaling and initiate transcription, thereby initiating cellular differentiation in general, and neuronal differentiation in particular. The present invention includes the use of polynucleotide sequences that code, entirely or partially, for the intracellular domain of Notch3, for the purpose of inducing cellular differentiation. The present invention includes the use of Notch3 intracellular domain polynucleotide or polypeptide sequences for the purpose of treating diseases or disorders, by inducing cellular differentiation.

### CROSS REFERENCE TO RELATED APPLICATIONS

This application is based on and claims priority to U.S. Provisional Application Ser. No. 62/033,058, filed Aug. 4, 2014, herein incorporated in its entirety.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB) SEQUENCE LISTING

The instant application contains a Sequence Listing, which has been submitted in ASCII format via EFS-Web and is hereby incorporated by reference in its entirety. Said ASCII copy, created on Aug. 3, 2015, is named N3ICD_ST25.txt and is 8838 bytes in size.

### STATEMENT REGARDING PRIOR DISCLOSURES BY THE INVENTOR OR A JOINT INVENTOR

Not Applicable

### BACKGROUND OF THE INVENTION

(1) Field of the Invention

The presently disclosed subject matter generally relates to the treatment of disorders in a subject, including but not limited to neurological disorders and cancer. More particularly, the methods of the presently disclosed subject matter relate to using the Notch3 receptor intracellular domain to induce cellular differentiation in proliferating cells, for the purpose of differentiating/generating/regenerating cells (e.g. neurons) or to inhibit tumor growth.

(2) Description of Related Art

Not Applicable

TABLE OF ABREVIATIONS

| | |
|---|---|
| AAV | adeno-associated virus |
| AD | Alzheimer's disease |
| ALS | Amyotrophic lateral sclerosis |
| AV | adenovirus |
| cDNA | complementary DNA |
| DCX | Doublecortin |
| Dlk1 | Delta-like 1 homologue |
| Dlk2 | Delta-like 2 homologue |
| DNA | deoxyribonucleic acid |
| DNER | Delta/notch-like epidermal growth factor-related receptor |
| E. coli | Escherichia coli |
| EMEM | Eagle's Minimum Essential Medium |
| GABA | gamma-aminobutiric acid |
| HD | Huntington's disease |
| HIV | human immunodeficiency virus |
| HSV | herpes simplex virus |
| iPSCs | induced pluripotent stem cells |
| LV | lentivirus |
| Mash1 | mammalian achaete-scute homolog 1 |
| ml | mililiter |
| N3 | Notch3 receptor |
| N3ICD | Notch3 intracellular domain |
| Ngn2 | Neurogenin 2 |
| PCR | polymerase chain reaction |
| PD | Parkinson's disease |
| PMSF | phenyl-methyl-sulphonyl-chloride |
| rAAV | recombinant adeno-associated virus |
| RNAi | RNA interference |
| SCA | spinocerebellar ataxia |
| SCI | spinal cord injury |
| SIV | simian immunodeficiency virus |
| TBI | traumatic brain injury |
| TetO | tetracycline operator sequence |
| TMD | trans-membrane domain |
| tTA | tetracycline transactivator protein |
| WT | wild-type |

### BACKGROUND

Notch receptors are transmembrane polypeptides that play a key role in organismal development, and are conserved from invertebrates to mammals. The divergent expression of Notch receptors and their ligands during development determines stem cell fate and dorso-ventral patterning, through a “lateral inhibition” mechanism. Defects in the expression or activation of Notch receptors or their ligands result in severe developmental abnormalities, especially in the nervous and cardiovascular systems. Up to the date of the submission of this invention, Notch receptors have been considered to be key inhibitors of neuronal differentiation and promoters of neural stem cells renewal and proliferation in the adult (Jan, et al., Annu Rev Genet. 28, 373-393 (1994); Artavanis-Tsakonas, et al., Science 284, 770-776 (1999)). In neural stem cells, Notch receptors, including Notch3, are known to promote the formation of astroglial cells (Tanigaki, et al., Neuron 29, 45-55 (2001)), which are themselves a type of neural progenitor cells (Doetsch, et al., Cell 97, 703-16 (1999)).

The Notch family includes 4 receptors (Notch1-Notch4) that can bind to any one of 5 “classical” ligands, Jagged 1, Jagged 2, Delta-like 1, 3 and 4, as well as to several atypical ligands including delta/notch-like epidermal growth factor-related receptor (DNER), contactin 1, contactin 6 and Delta-like 1 and 2 homologues (Dlk1, 2). Ligand binding initiates sequential Notch receptor cleavage, releasing the intracellular receptor domain, which migrates to the nucleus and initiates gene transcription, as a transcriptional co-activator. All 4 Notch receptors are currently thought to have similar functions in promoting cell proliferation.

Neurodegenerative disorders affect a large proportion of the population, resulting in severe mental and/or physical impairment and eventually death. There is a wide spectrum of neurodegenerative disorders including but not limited to Alzheimer's disease (AD), Parkinson's disease (PD), Huntington's disease (HD), spinocerebellar ataxia (SCA), prion protein disease, amyotrophic lateral sclerosis (ALS) and aging-related neuronal death. These diseases are often caused by genetic factors or protein misfolding, however their mechanisms are not fully understood and usually there is no treatment available. Neurodegeneration also occurs after central nervous system injury, for example in cases of spinal cord injury (SCI), traumatic brain injury (TBI), stroke, hypoxia, alcoholism, and other conditions, for which there is also no treatment.

Stem cell replacement therapy has emerged as a promising method to replace cell (e.g. neurons) lost to disease or injury (Lunn, et al., Ann Neurol. 70, 353-61 (2011)). PD was the first disorder to be treated with fetal tissue transplants (Freed, et al., Prog Brain Res. 82, 715-21 (1990)), which was later replaced with embryonic stem cells (Kim, et al., Nature 418, 50-56 (2002)) and induced pluripotent stem cells (iPSCs) (Skalova, et al., Int J Mol Sci. 16, 4043-67 (2015)). Similar attempts have been made in spinal cord repair (Suzuki, et al., Trends Neurosci. 31, 192-8 (2008); Papadeas, et al., Curr Opin Biotechnol. 20, 545-51 (2009); Teng, et al., Sci Transl Med. 4, 165 (2012); Hefferan, et al., PLOS One 7, e42614 (2012)). Stem cell transplants are currently under clinical trials for ALS (Boulis, et al., Nature Reviews Neurology 8, 172-176 (2012); Glass, et al., Stem Cells 30, 1144-51 (2012)) and in advanced animal research stages for SCI (Abematsu, et al., J Clin Invest. 120, 3255-66 (2010); Nori, et al., PNAS 108, 16825-30 (2011)). However, stem cell transplants combined with growth factor or pharmacological treatments only moderately and transiently delay disease progression (Teng, et al., Sci Transl Med. 4, 165 (2012); Glass, et al., Stem Cells 30, 1144-51 (2012)). In addition, stem cell transplants require immunosuppressive therapy. The alternative autologous transplantation of iPSCs eliminates the requirement for immunosuppressive therapy but propagates the same genetic defects present in the degenerating neurons and also presents the risk of teratomas in the absence of strict cell selection (Tsuji, et al., PNAS 107, 12704-9 (2010)). By either method, only a small fraction of transplanted stem cells become mature neurons, distributed only locally at the transplant site (Hefferan, et al., PLOS One 7, e42614 (2012)). Moreover, the adult nervous system environment favors the differentiation of exogenous stem cells predominantly into glial cells (Shihabuddin, et al., J. Neurosci. 20, 8727-35 (2000); Hugnot, et al., Frontiers Biosci. 16, 1044-59 (2011)). Therefore symptom improvements observed using stem cell therapy result mostly from the protective effect of stem cell- or glia-secreted growth factors rather than from newly generated neurons (Horner, et al., J. Neurosci. 20, 2218-28 (2000); Chi, et al., Stem Cells 24, 34-43 (2006); Barnabe-Heider, et al., Cell Stem Cells 7, 470-482 (2010); Hefferan, et al., PLOS One 7, e42614 (2012); Hugnot, et al., Frontiers Biosci. 16, 1044-59 (2011)). Methods to induce the differentiation of neural progenitor cells into viable, functionally integrated neurons, with the high efficiency needed to generate clinically significant results, have not been developed yet. The current invention provides a method of generation of differentiated cells, including new neurons, for this unmet medical need.

Cancer is generally defined as a disease produced by uncontrolled cellular proliferation and invasion. This cellular proliferation process occurring in the adult is similar to the proliferation that occurs during embryonic development and involves cell signaling through Notch receptors. This role is supported by the fact that some tumors, including nervous system tumors, show increased expression of Notch receptors, including Notch3 (Bellavia, et al., EMBOJ. 19: 3337-48 (2000); Dang, et al., Dev Neurosci. 28: 58-69 (2006); Pierfelice, et al., Cancer Res. 71: 1115-25 (2011)). As a result, the inhibition of Notch, including Notch3 signaling has been considered a potential method to treat cancer (van Es, et al., Trends Mol Med. 11:496-502 (2005); Rahman, et al., Am J Clin Pathol. 138: 535-44 (2012)). The current invention demonstrates that Notch3 may in fact have the opposite effect, promoting differentiation of tumor cells, and providing a potential treatment method for this unmet medical need.

Notch 1-4 have been generally considered to have similar functions, however each Notch receptor shows some structural and functional particularities. Notch3 is more than 200 amino-acids shorter than Notch1, missing several structural domains present in Notch1. For example a transactivation domain present in the intracellular segment of the Notch1 receptor is missing in Notch3. As a result, Notch3 is a weaker transcriptional inducer relative to Notch1 (Shimizu, et al., Biochem Biophys Res Commun. 291:775-9 (2002) and can act as a repressor of Notch1-mediated transcription (Beatus, et al., Development 126: 3925-3935 (1999).

### BRIEF SUMMARY OF THE INVENTION

The present invention describes the use of the intracellular domain of Notch3 receptor (N3ICD) and sub-domains thereof to promote cellular differentiation, including the production of neurons in-vitro and in-vivo, and of other cell types, from undifferentiated cells. Another aspect of the invention includes the use of N3ICD and sub-domains thereof to induce the differentiation of tumor cells.

The invention includes the polynucleotide sequence of N3ICD (SEQ ID NO:1) and the corresponding amino-acid sequence (SEQ ID NO:2). Another embodiment of this invention comprises corresponding N3ICD sequences in all vertebrate and invertebrate animals. Another embodiment of this invention comprises sub-domains of the N3ICD sequences and variations thereof, which maintain functional similarity with human N3ICD, and which could be used in medical applications with a similar outcome.

Another embodiment of the present invention includes the cells and vectors harboring the N3ICD sequences of the present invention, including the use of N3ICD, and variations thereof, in gene therapy.

Another embodiment of the present invention includes the use of N3ICD polypeptide, and subdomains thereof, for direct introduction into cells for the purpose of inducing cellular differentiation, including the generation of new neurons.

Another embodiment of the present invention includes alternative methods of specifically generating the N3ICD sequence in-vivo, using antibodies, ligands or protease activators, for the purpose of inducing cellular differentiation, including the generation of new neurons.

Another embodiment of the present invention includes the use of cells engineered to express the N3ICD for the purpose of regenerating or generating new neurons, for the treatment of neurological disorders, including neurodegenerative disorders and accidental neuronal damage.

Another embodiment of the present invention includes the use of N3ICD, and variations thereof, in the treatment of neurological and psychiatric disorders associated with defective or incomplete neuronal differentiation.

Another embodiment of the present invention includes the use of cells and vectors engineered to express Notch3 intracellular domain or variations thereof in the treatment of cancer.

Another embodiment of the present invention includes the use of cells and vectors engineered to express Notch3 intracellular domain or variations thereof in the generation of neuronal networks on an artificial or natural support, scaffold, tissue, organ, or electronic circuit.

Another embodiment of the present invention includes the use of Notch receptor or ligand domains, antibodies or pharmacological compounds to elicit the intracellular expression of N3ICD or variations thereof for the purpose of generating neuronal networks on an artificial or natural support, scaffold, tissue, organ or electronic circuit.

### BRIEF DESCRIPTION OF THE FIGURES

FIG. 1 depicts the general location of the Notch3 intracellular domain (N3ICD). N3ICD includes the entire intracellular region of the Notch3 receptor, from the end of its transmembrane domain (TMD) to the C-terminus of Notch3 receptor.

FIG. 2 (A-F) depicts the complementary DNA (cDNA) nucleotide sequences of N3ICD in three mammalian species (human, mouse, rat) and a fish species (zebrafish), with corresponding sequences aligned. Shown in the figure are the nucleotide positions corresponding to their location in the full-length cDNA of each gene. For example, the human N3ICD cDNA sequence (SEQ ID NO:1) extends from nucleotide 5062 to nucleotide 7044 of the human Notch3 cDNA, as numbered in gene databases commonly known to those of ordinary skill in the art. Accession numbers for human, mouse, rat and zebrafish Notch3 cDNA are indicated in brackets, corresponding to the identity of these genes in gene databases. The cDNA sequences for human, mouse and rat N3ICD are 83.09% identical. Non-identical nucleotides between human, mouse and rat N3ICD are indicated by asterisk (*). The cDNA sequence for zebrafish N3ICD is 31.1% identical with corresponding mammalian sequences. Non-identical nucleotides between zebrafish and mammalian N3ICD are indicated by hashtag (#).

FIG. 3 (A-B) depicts the amino-acid sequences of N3ICD in three mammalian species (human, mouse, rat) and a fish species (zebrafish), aligned corresponding to the nucleotide sequences shown in FIG. 2. The numbers indicate the amino-acid positions respective to the full length Notch3 proteins. For example, human N3ICD (SEQ ID NO:2) is located between positions 1662 and 2321 of Notch3. Human, mouse, rat and zebrafish N3ICD have 60.6% identity and 68.5% similarity. Identical amino-acid sequences are underlined.

FIG. 4 depicts Western blot experiments showing the protein expression levels of Notch3, Notch1 and proneural markers Mammalian achaete-scute homolog 1 (Mashl), Neurogenin 2 (Ngn2) and Doublecortin (DCX) in neuroblastoma Neuro-2a cells. Protein expression is shown in Neuro-2a cells after 5 days in culture, under non-differentiating conditions, where the culture medium contains 10% fetal bovine serum (+FBS), or under differentiating conditions, where the fetal bovine serum is removed from the cell culture medium (−FBS). Notch3 expression is increased under differentiating conditions, in opposition to Notch1, but in correlation with the neuronal markers, indicating the specific expression of Notch3 in neuronal cells. Beta-actin is used as a concentration standard.

FIG. 5 (A-C) depicts the effect of N3ICD expression on the differentiation of the neuroblastoma cell line Neuro-2a. FIG. 5A depicts the differentiation of Neuro-2a cells in culture. Unmodified, wild-type Neuro-2a cells (WT) proliferate when grown in medium containing 10% fetal bovine serum (+FBS), and differentiate into neurons when FBS is removed from the culture medium (−FBS). Neuro-2a cells engineered to express exogenous N3ICD differentiate even under non-differentiating conditions (+FBS). Cells where Notch3 expression was eliminated using the RNA interference technology (N3-RNAi), do not differentiate even under differentiating conditions (−FBS). FIG. 5B depicts Western blots showing the cellular expression of exogenous N3ICD after introduction in Neuro2a cells (upper blot), and the reduced expression of Notch3 receptor after RNAi treatment of Neuro-2a cells (lower blot). FIG. 5C depicts a bar graph showing the quantification of cellular differentiation under various conditions, which was determined by counting the fraction of cells with neurite extensions of at least twice the length of cell body relative to the total number of cells.

FIG. 6 depicts Western blot experiments showing the variation of protein levels of neuronal marker Ngn2 as a function of Notch3 expression, in WT, N3ICD or N3-RNAi cells, as defined in FIG. 5. WT cells cultured in differentiating (−FBS) conditions for 5 days show an increase in Ngn2 protein level, which correlates with the neurite growth associated with neuronal differentiation depicted in FIG. 5. The artificially induced expression of N3ICD increases Ngn2 expression by approximately 50%. In contrast, the inhibition of Notch3 expression by RNAi (N3-RNAi) reduces Ngn2 expression to negligible levels.

### DETAILED DESCRIPTION

In subjects with particular disorders or disabilities, including neurological, sensory and psychiatric disorders and cancer, alterations in cellular numbers and/or activity can occur. Accordingly, by providing subjects suffering from such disorders with new cells that replace the cells, e.g. neurons, lost to disease or injury, cellular (e.g. neuronal) regeneration can alleviate or eliminate such disorders or disabilities. As disclosed for the first time herein, cells engineered or induced to express N3ICD can differentiate in-vivo and in-vitro, to generate cells with similar functional characteristics as the cells lost to injury or disease, and which can functionally replace such damaged cells.

Another embodiment of the present invention includes the use of cells and/or vectors engineered to express N3ICD in the treatment of disorders resulting from incomplete or defective cellular differentiation, including cancer and some sensory and psychiatric disorders, including but not limited to chronic pain, schizophrenia and bipolar disorder. In such patients, gene therapy using cells or vectors engineered to express N3ICD to induce the differentiation of incompletely differentiated or of proliferating cells, can alleviate or eliminate such disorders.

All publications mentioned herein are incorporated herein by reference to the extent allowed by the law for the purpose of describing and disclosing the proteins, vectors, cells and methodologies reported therein that might be used with the present invention. However, nothing herein is to be construed as an admission that the invention is not entitled to antedate such disclosure by virtue of prior invention.

### DEFINITIONS

This invention is not limited to the methods, protocols, cell lines, vectors or reagents described herein because they may vary. Furthermore, the terminology used herein is for the purpose of describing particular embodiments only and is not intended to limit the reach of the present invention. Although any materials and methods similar or equivalent to those described herein can be used in the practice of the present invention, representative materials and methods are described herein.

Following patent law convention, the terms “a”, “an”, and “the” refer to “one or more”, e.g. reference to “a cell” includes a plurality of cells. Unless defined otherwise, all technical and scientific terms used herein have the same meanings as commonly understood by one of ordinary skill in the art in the field of the invention.

Unless otherwise indicated, all numbers expressing quantities of ingredients, conditions, and so forth used in the specification and claims are to be understood as being modified in all instances by the term “about”. Accordingly, the numerical parameters set forth in the current specification are approximations that can vary depending on the desired properties sought by the presently disclosed subject matter. Furthermore, Applicants desire that the following terms be given the particular definition as defined below.

The term “Notch3” shall be construed as including proteins from any species, as well as artificial amino-acid sequences that maintain sequence or functional similarity, or both, to human Notch3 (SEQ ID NO:1), as typically understood by those of ordinary skill in the art, regardless of whether the candidate protein is named “Notch3” or not. Furthermore, the term “Notch3” shall be construed to also include nucleic acid sequences, including DNA and RNA sequences equivalent to the amino-acid sequence of Notch3.

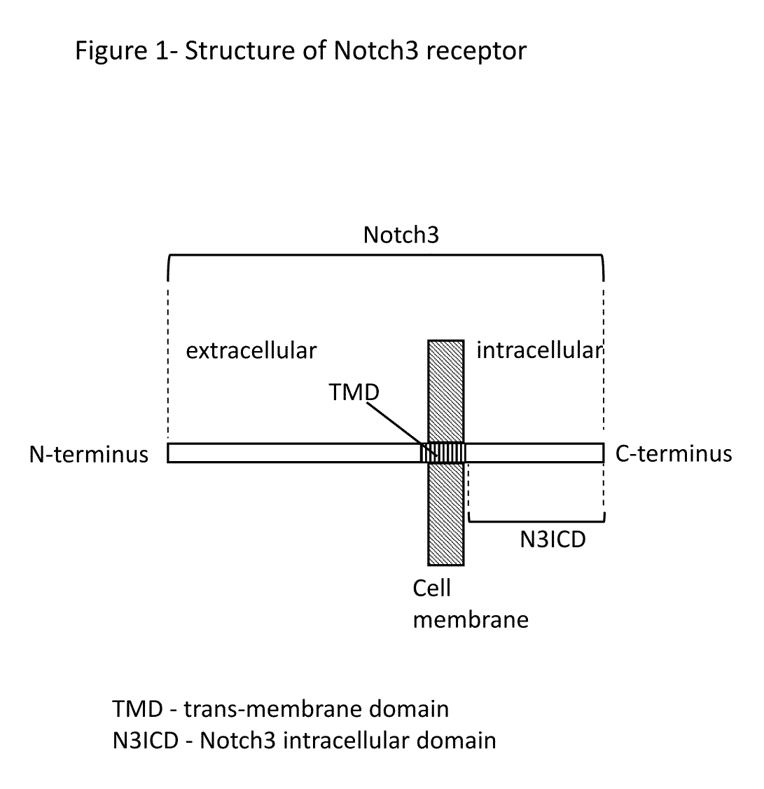

The term “Notch3 intracellular domain” (N3ICD) shall be construed as representing the fragment of Notch3, including the amino-acid sequence and the corresponding nucleotide sequence, located between the transmembrane domain (TMD) and the C-terminus of Notch3, including sub-domains thereof, as represented in FIG. 1. For example, human N3ICD represents the amino-acid sequence located between amino-acids 1662-2321 of human Notch3 (SEQ ID NO:2). Because of the degeneracy of the genetic code, a multitude of nucleotide sequences encoding N3ICD, or fragments thereof, may be used. Nucleotide sequences may vary by selecting nucleotide combinations based on possible codon choices, in accordance with standard triplet genetic codes.

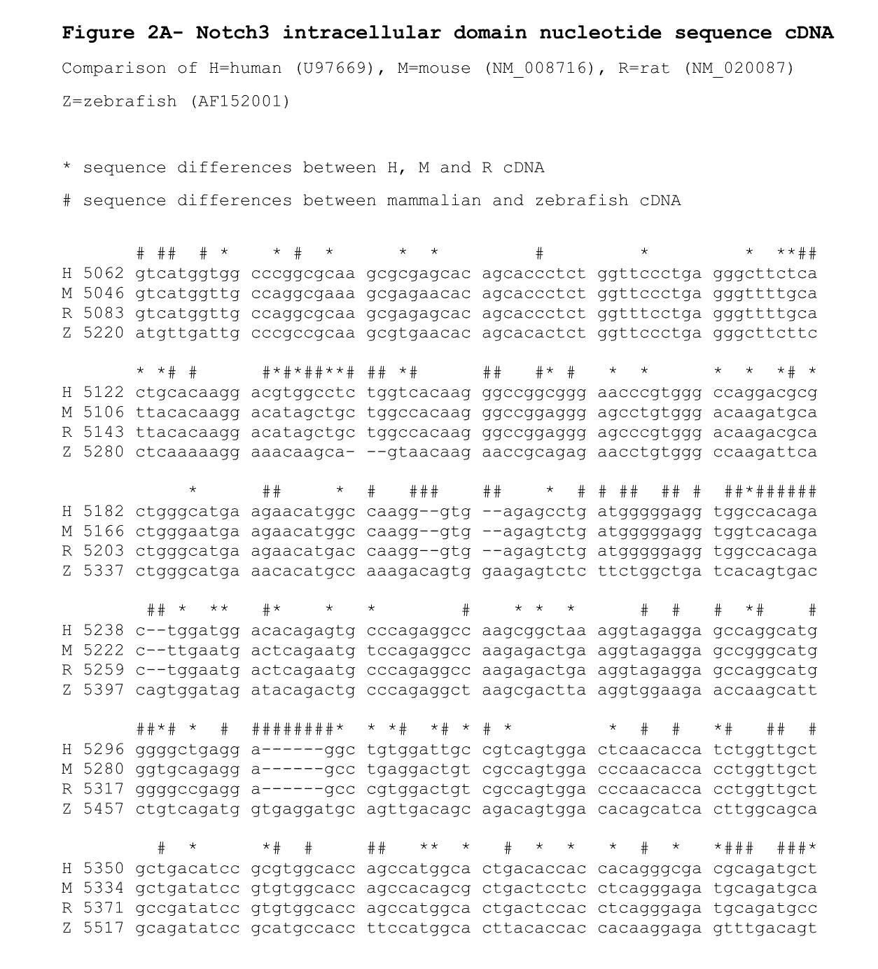

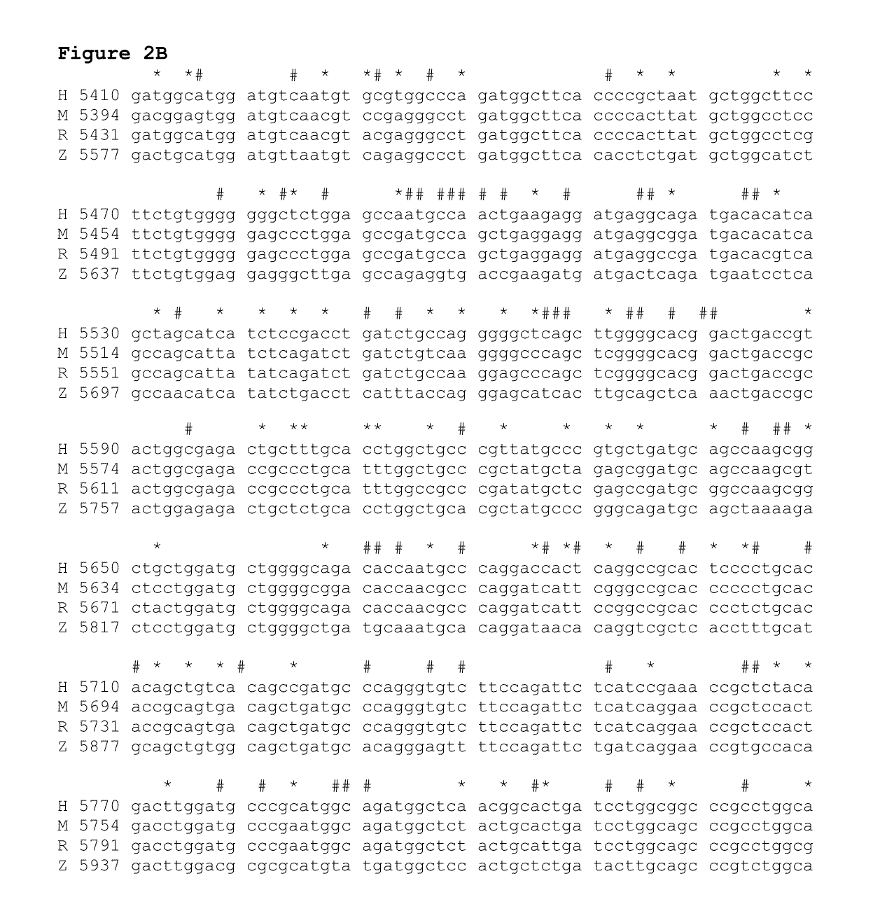

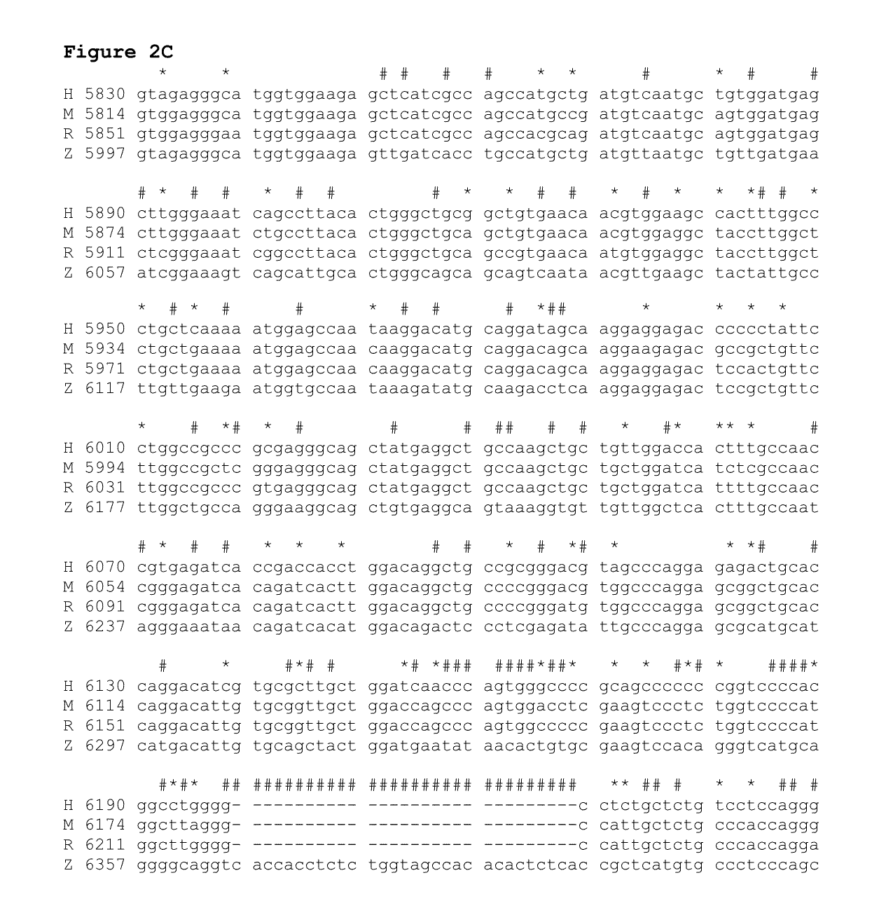

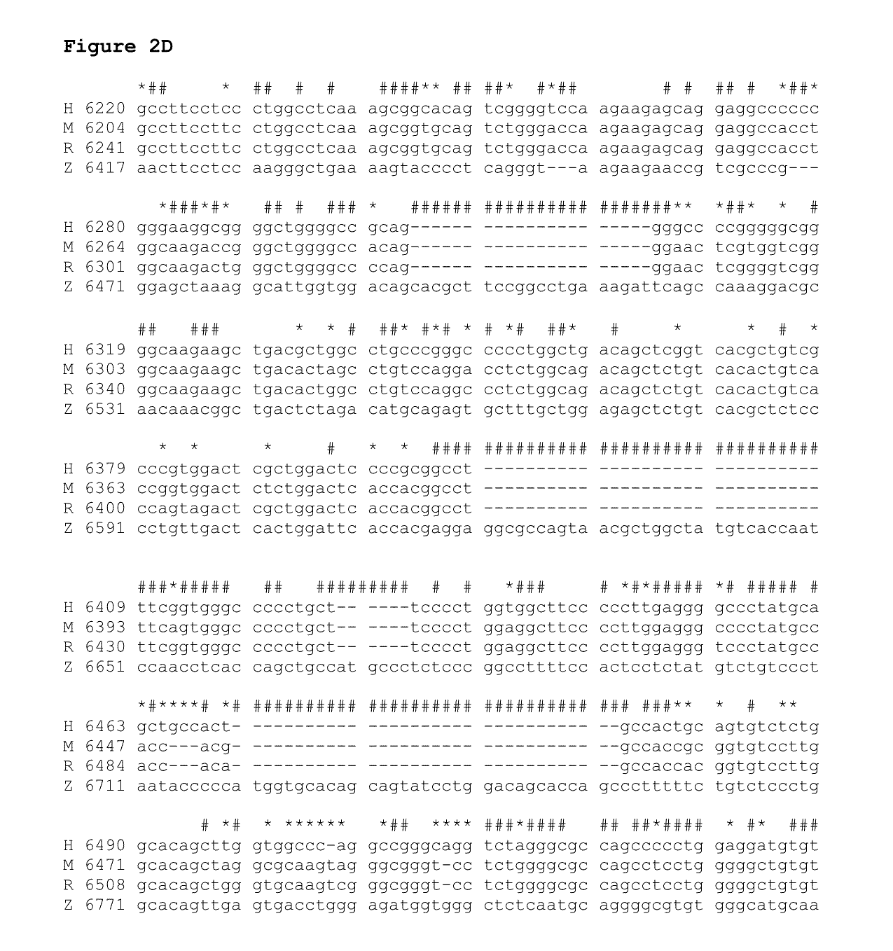

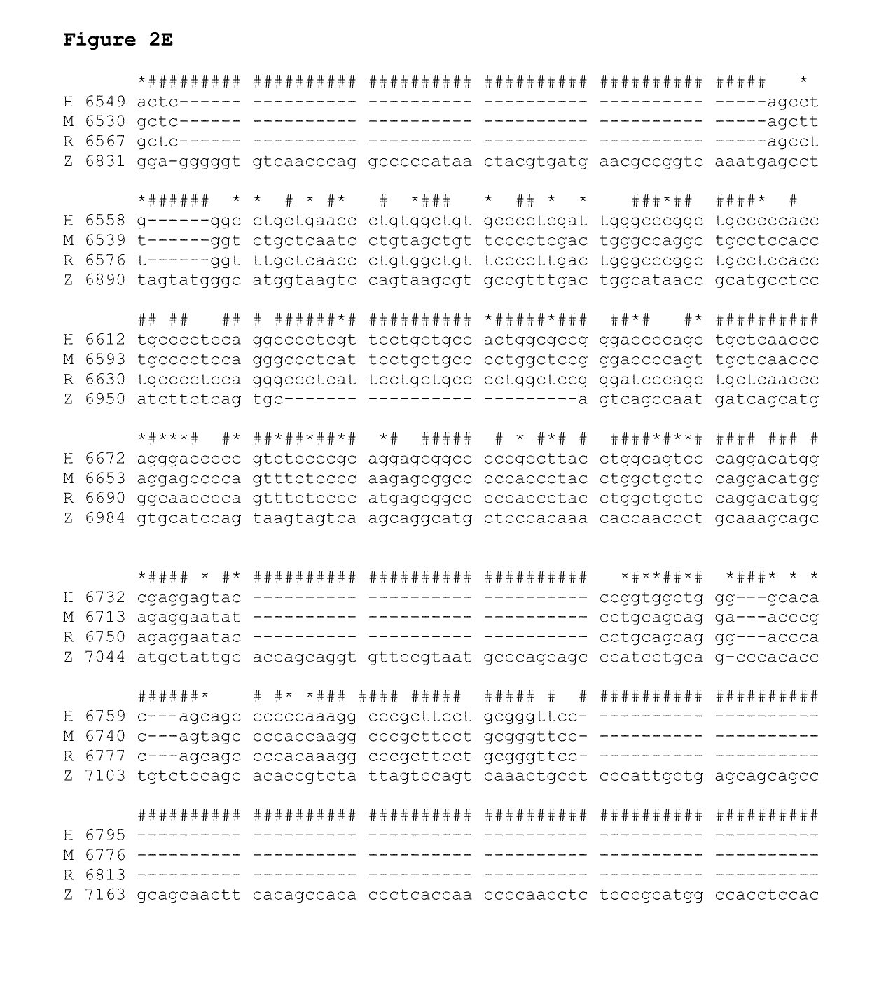

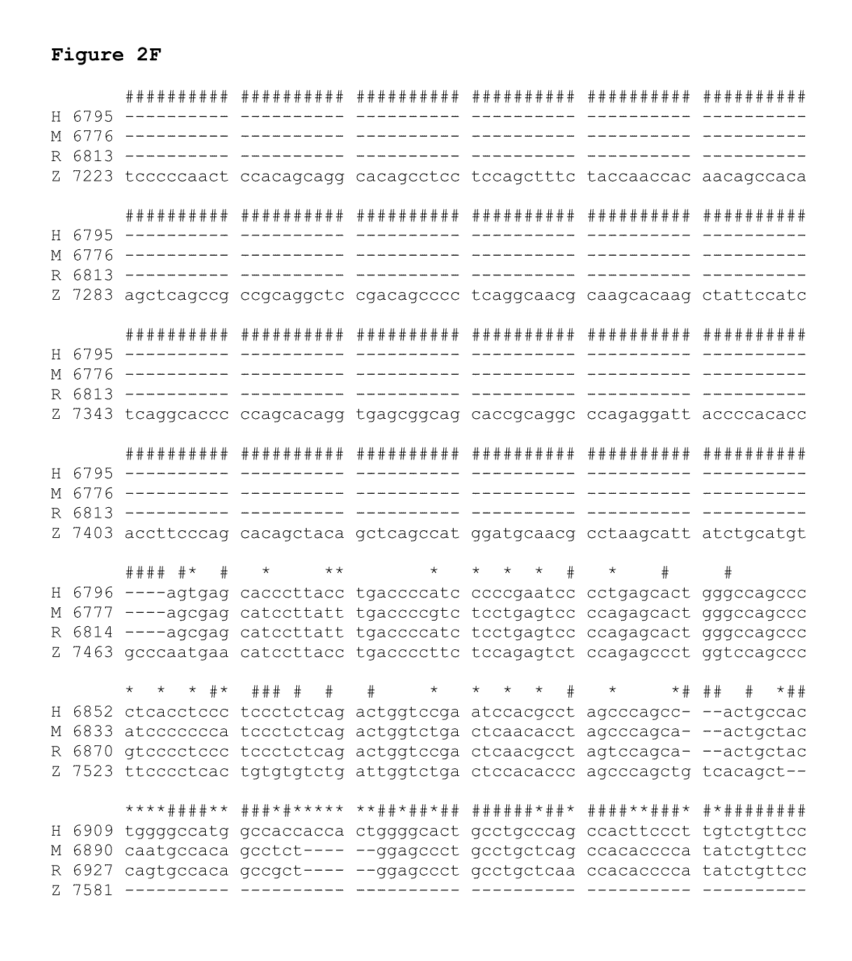

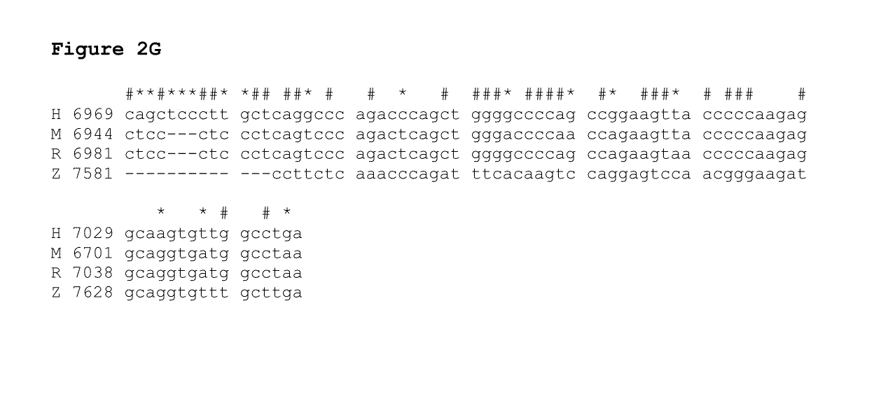

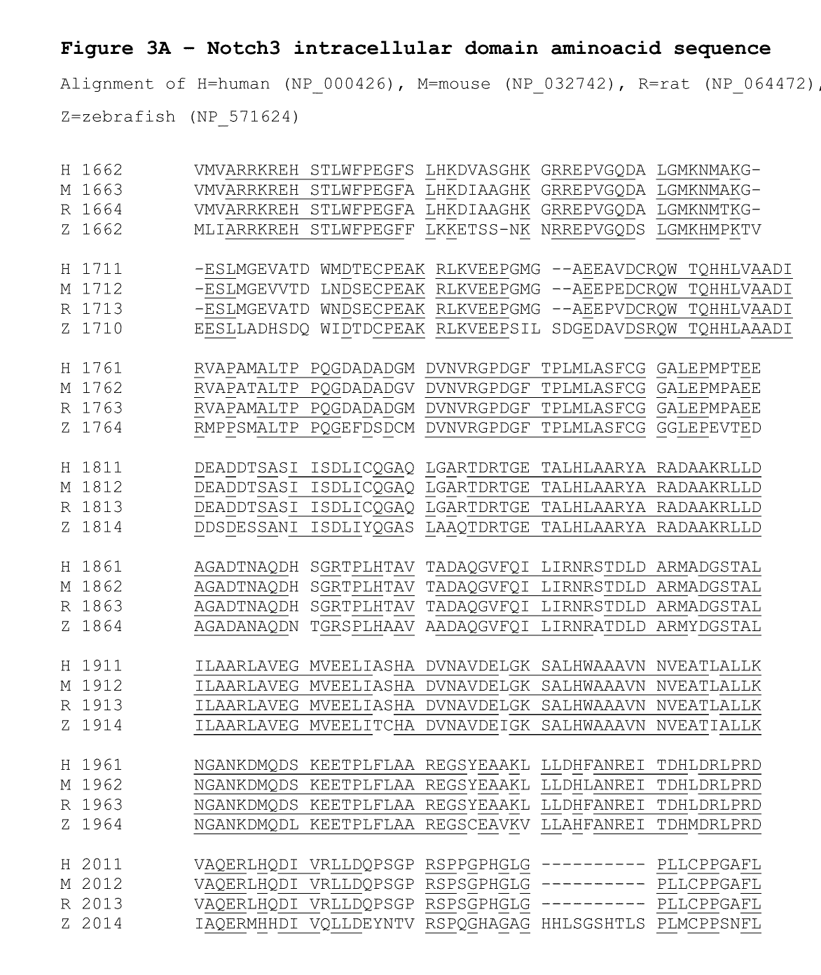

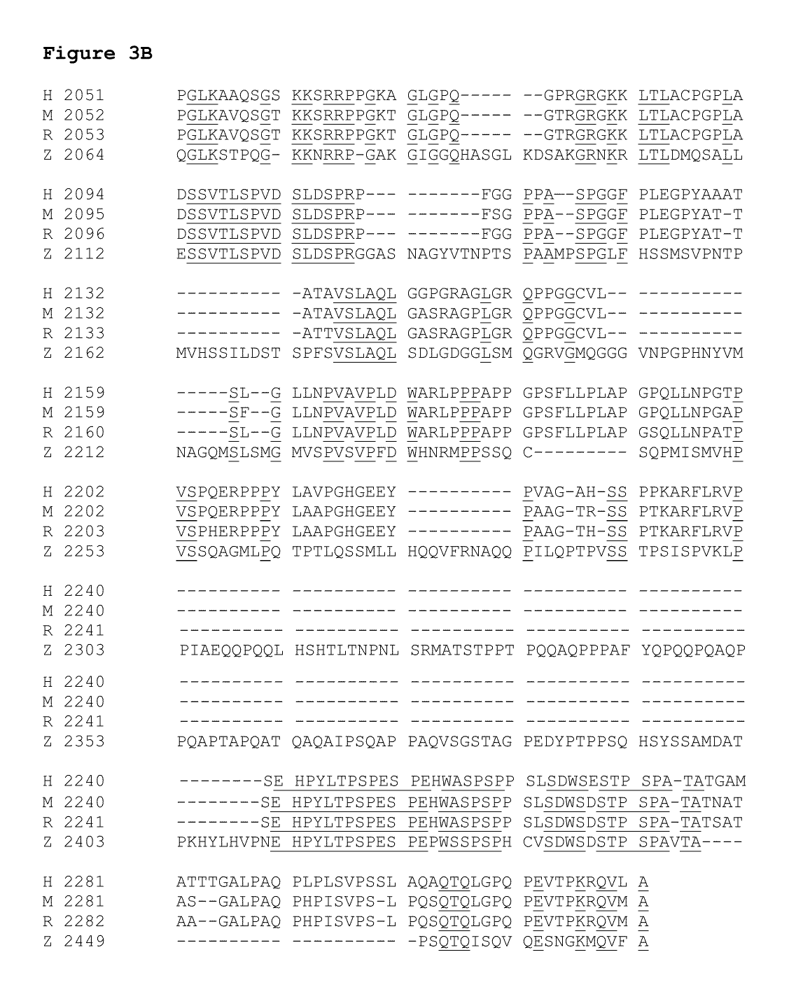

The terms “domain” and “sub-domain” as used herein shall be construed as representing amino-acid or nucleotide sequences that are identical or similar to parts of the N3ICD amino-acid or corresponding nucleotide sequences after aligning the sequences and introducing gaps if necessary to achieve the maximum percentage identity for the entire sequence. The alignment of three mammalian (human, mouse and rat) and one fish (zebrafish) N3ICD cDNAs is depicted in FIG. 2 (A-F), showing the conservation of many regions of the nucleotide sequence, across different species. Because of the degeneracy of the genetic code, human mouse and rat N3ICD cDNA sequences are 83.09% identical, but correspond to amino-acid sequences that are 94.82% identical and 97.26% similar, as depicted in FIG. 3 (A-B). Although the zebrafish N3ICD cDNA has only approximately 31% identity with corresponding mammalian sequences, the regions that are identical code for conserved domains of N3ICD amino-acid sequence that may preserve essential functional properties (underlined in FIG. 3 A-B). Examples of such conserved domains, which are deemed to be important for the function of N3ICD, include amino-acid sequences 1665-1715, 1723-1737, 1746-1761, 1764-2026, 2041-2054, 2092-2109, 2135-2140, 2240-2276 and 2304-2318 corresponding to human Notch3.

Alternatively, a gene or cDNA sequence encoding N3ICD (SEQ ID NO:1), or fragments thereof, may be cloned into an expression vector or plasmid, and expressed in any of a number of expression systems according to methods well known to those of ordinary skill in the art.

The phrase “functionally similar” with respect to amino-acid or nucleotide sequences shall be construed to represent amino-acid sequences, or nucleotide sequences equivalent to such amino-acid sequences, that have a similar effect on cell physiology as N3ICD (SEQ ID NO:1 and SEQ ID NO:2), with typical meaning for those of ordinary skill in the art. The term shall not be construed to be limited to specific percentage ranges of sequence identity or similarity with N3ICD, as the introduction of gaps, insertions, substitutions or extensions in an amino-acid or nucleotide sequence may substantially change the percentage of sequence identity or similarity, but may maintain the same functionality as the original N3ICD sequence.

The phrase “similar effect on cell physiology as N3ICD” shall be construed as meaning the induction of transcription of the same or similar genes as induced by N3ICD, resulting in a similar process of cellular differentiation, as typically understood by one of ordinary skill in the art.

The term “identity” or “homology” shall be construed to mean the percentage of the amino-acid residues in the candidate sequence that are identical with the residues of a corresponding sequence to which it is compared, after aligning the sequences and introducing gaps if necessary to achieve the maximum percentage identity for the entire sequence, and not considering any conservative substitutions as part of the sequence identity. Neither insertions, deletions nor extensions shall be construed as reducing sequence identity or homology. Methods and computer programs for sequence alignment are well known in the art. Sequence identity may be measured using sequence analysis software.

The term “similarity” shall be construed as meaning the percentage of the amino-acid residues in the candidate sequence that are similar with the residues of a corresponding sequence to which it is compared, after aligning the sequences and introducing gaps if necessary to achieve the maximum percentage identity for the entire sequence.

The term “similar” in reference to amino-acids shall be construed as meaning amino-acid residues in the candidate sequence that have similar physico-chemical properties as the amino-acid residues located at the same position of a corresponding sequence to which it is compared, after aligning the sequences and introducing gaps if necessary to achieve the maximum percentage identity for the entire sequence. Similar physico-chemical properties may include comparisons between amino-acids with respect to amino-acid acidity, basicity, hydrophobicity, hydrophilicity, molecular size, aromaticity or other properties.

The terms “cell”, “cell line” and “cell culture” include progeny. It is understood that all progeny may not be precisely identical in DNA or protein content, due to deliberate or accidental mutations. Variant progeny that have the same function or biological property as determined in the originally characterized cell, are included. The cells used in the present invention are generally eukaryotic or prokaryotic cells.

The term “vector” shall be construed as meaning a DNA or RNA sequence which is functionally linked to a suitable polynucleotide control sequence capable of producing the expression of the DNA in a cell. Such control sequences include a promoter to initiate transcription, an optional operator sequence to control transcription, an origin of replication, a cloning site, selectable markers, a sequence encoding RNA ribosome binding sites, and sequences that control the termination of transcription and translation. The vector may be a plasmid, a phage or virus particle, a cosmid, an artificial chromosome, or a genomic insert. After introduction in a cell, the vector may replicate and function independently of the cell genome, or may in some cases integrate into the genome itself. In the present specification, “vector” and “plasmid” may be used interchangeably, as the plasmid is the most commonly used form of vector. However, the invention is intended to include other forms of vectors which serve equivalent function and which are known in the art.

Alternatively, a vector may include, in addition to the elements described above, an inducible promoter, which activates gene expression only under specific, controllable conditions. Such controllable conditions include a specific temperature (e.g. heat shock promoter), a specific chemical (e.g. doxycycline, dexamethasone, etc.), or other conditions.

The terms “transformation”, “transfection” and “infection” shall be construed as meaning the introduction of a vector containing a polynucleotide sequence of interest into a suitable cell, whether or not any coding sequences of that vector are expressed. The cell where the vector is introduced is termed “host cell”. The introduced polynucleotide sequence may be from the same species as the host cell, from a different species, or may be a hybrid polynucleotide sequence containing sequences from both the same species and a different species than the host cell. Methods of transfection include electroporation, calcium phosphate, liposome, DEAE-dextran, microinjection, polybrene, and others. The term “infection” shall be construed as meaning a transfection by use of a viral vector. Examples of viral vectors include adenovirus (AV), adeno-associated virus (AAV), lentivirus (LV), herpes simplex virus (HSV), simian immunodeficiency virus (SIV), human immunodeficiency virus (HIV) and others.

In addition to the above definition, the term “transfection” shall be construed to also include the introduction of a protein into a host cell. Protein transfection may be achieved using a variety of commercially available reagents and kits, e.g. cationic lipid mixtures, peptides, etc.

The term “mammal” shall be construed as including any animal classified as a mammal according to established and published rules that are well known to those of ordinary skill in the art. The term “animal” is construed as including any living organism characterized by established and published classifications that are well known to those of ordinary skill in the art.

The term “stem cell” shall be construed as including cells that maintain the ability to become any type of cell that is present in an organism. Examples of stem cells include embryonic stem cells, mesenchymal stem cells, amniotic stem cells, dental pulp stem cells, induced pluripotent stem cells, and others. The term “progenitor cell” is construed as including cells that maintain the capability of becoming a subset of all the cell types present in an organism. For example, a neural progenitor cell is a progenitor cell that can become one of several types of cells present in the nervous system. In the present specification, “stem cell” and “progenitor cell” may be used interchangeably, as the progenitor cell is a type of stem cell that has acquired some individual characteristics that differentiate it from a stem cell. For example, a neural progenitor cell may express a partially different subset of genes than a stem cell, which limit the ability of the neural progenitor cell to become only a cell type present in the nervous system. However, it is understood that both stem cells and progenitor cells are continuously dividing cells, and produce through division daughter cells identical to the dividing parent cell, over a large number of divisions. Alternatively, cells with stem cell properties may be derived from natural sources (e.g. cancer cells).

The term “cellular differentiation” shall be construed as representing the process by which a stem cell or a progenitor cell ceases to divide, and begins to acquire physical and functional characteristics that are different from the physical and functional characteristics of the cell from which it was produced, and from other cell types. A completely or fully differentiated cell is a cell that has reached a state characterized by a maximal, final functional role in comparison to the other cells with a similar phenotype present in an organism. For example, a fully differentiated neuron is a cell that expresses a typical set of genes, has a typical electrophysiological response and performs a typical physiological function, usually as part of a cellular network, as understood by those of ordinary skill in the art.

A cell may present various degrees or levels of differentiation, which is a continuous, not a punctual process. For example, progenitor cells display some degree of differentiation relative to stem cells, as progenitor cells can produce by differentiation only a subset of the cells that stem cells can produce. However, progenitor cells maintain the ability to divide into identical daughter cells, similar to stem cells. Immature cells are construed as including cells that are characterized by an incomplete degree of differentiation. In addition, immature cells may display some phenotypical and physiological characteristics similar to the characteristics of un-differentiated cells, some characteristics similar to the characteristics of fully differentiated cells, as well as some unique characteristics that are different from both un-differentiated and fully differentiated cells. For example, immature neurons are neurons that express a subset of the genes typically expressed in neural progenitor cells, as well as some of the genes expressed in fully differentiated neurons, and a set of genes that are expressed neither in neural progenitor cells, nor in fully differentiated neurons. In addition, immature neurons display unique electrophysiological properties, as commonly known to those of ordinary skill in the art. For example immature neurons produce an electric response when stimulated with the amino acid gamma-aminobutiric acid (GABA), which reflects the presence of GABA receptors and ion channel proteins similar to neurons, however this electric response produced has the opposite sign relative to the electric response produced by fully differentiated or mature neurons.

As used herein, the terms “neurodegenerative disorder” and “neurological disorder” may be used interchangeably, and include any disorder characterized by damage to nervous system cells, and include the following, without limitation, Alzheimer's disease, Huntington's disease, Parkinson's disease, amyotrophic lateral sclerosis, epilepsy, multiple sclerosis, prion protein disease, spinal cord injury, traumatic brain injury, stroke, ischemia, hypoxia, diabetic neuropathy, peripheral neuropathy, peripheral nerve injury, glaucoma, retinal degeneration, auditory nerve degeneration, spinocerebellar ataxia (SCA), and aging-related or chemically-related neuronal death.

The term “cancer” as used herein, is construed as including any disorder characterized by abnormal and uncontrolled cellular proliferation of some cells in the human body, resulting in damage to other cells and organs in the body, and leading to a state of illness.

### Methods for Producing the Notch3 Intracellular Domain (N3ICD)

The invention provides polynucleotides or nucleic acids, e.g. complementary DNA (cDNA), comprising a nucleotide sequence encoding N3ICD SEQ ID NO:1, or fragments thereof. The invention also encompasses nucleotide sequences that code for amino-acid sequences that are functionally similar to N3ICD.

The polynucleotides may be obtained by any method known in the art. For example, a nucleotide sequence of N3ICD may be assembled from chemically synthesized oligonucleotides or by duplication of a naturally occurring N3ICD sequence. N3ICD polynucleotides obtained through any methods may be further amplified by PCR, using synthetic primers hybridizable to the 3′ and 5′ ends of the sequence, cloned into a replicable vector using any method well-known in the art, and amplified in a host cell.

In another embodiment, the invention further provides polypeptides, comprising amino-acid sequences corresponding to N3ICD, or fragments thereof. The invention also encompasses amino-acid sequences that are functionally similar to N3ICD.

The polypeptides may be obtained by any method known in the art. For example an amino-acid sequence of N3ICD may be assembled from chemically synthesized amino-acids or by expression from a corresponding cDNA sequence.

The nucleotide sequence and corresponding amino-acid sequence of N3ICD, and fragments thereof, may be manipulated using methods well known in the art for the manipulation of nucleotide sequences, e.g. recombinant DNA techniques, site directed mutagenesis, PCR, etc. (Sambrook, et al., Molecular Cloning, A Laboratory Manual, 2^{nd} ed., Cold Spring Harbor Laboratory (1990), incorporated by reference herein in its entirety), to generate polypeptides having different amino-acid sequences, for example to generate amino-acid substitutions, insertions and/or deletions.

The Notch3 intracellular domain (N3ICD) complementary DNA (cDNA) can be generated from full-length Notch3 cDNA or from genomic DNA by polymerase chain reaction (PCR), using a primer complementary to the 3′ end of the DNA sequence that includes the N-terminal end of N3ICD, and a second primer complementary to the 3′ end of the DNA sequence that includes the C-terminal end of N3ICD, as well as a DNA polymerase and a mixture of deoxynucleoside triphosphates. For example, in humans the N-terminal end of N3ICD corresponds to nucleotide number 5071 of the full length Notch3 cDNA (accession number U97669), and the C-terminal end of N3ICD corresponds to nucleotide number 7044 of the full length Notch3 cDNA (FIG. 2). Each primer also includes a restriction recognition site immediately outside of the DNA region of interest, where the DNA molecule can be cut by restriction enzymes in order to preserve only the DNA fragment of interest. Examples of restriction enzymes include, but are not limited to EcoRI, BamHI, HindIII, NotI, and others. The DNA fragment of interest corresponding to N3ICD cDNA can be then separated by electrophoresis on a separation gel, capillary or through other separation methods. Gels commonly used for DNA purification include agarose, polyacrylamide, and other gels.

The N3ICD cDNA can be introduced, using a DNA ligase enzyme, in a suitable vector that was previously cut at the cloning site with the same restriction enzymes that were used to obtain the N3ICD cDNA. The DNA ligase enzyme helps bind the ends of the N3ICD cDNA fragment to the matching ends of the vector DNA. The success of the insertion can be verified by analyzing the DNA ligation mixture by electrophoresis, in parallel with the original vector. The vector containing the N3ICD insert can be separated from the gel, purified and then can be introduced in a host cell for amplification in cell culture.

The host cells containing the vector that includes the N3ICD cDNA can be multiplied in cell culture in a medium that includes a selection chemical compound, for example ampicillin, chloramphenicol, gentamycin, or others. Examples of host cells include bacterial cells (e.g. E. coli), yeast, insect cells, mammalian cells and others. After amplification, the vector containing N3ICD cDNA is extracted from the host cells. Extraction methods may vary and may include host cell lysis, DNA precipitation, centrifugation, liquid-liquid extraction, gel filtration, other types of chromatographic separations, and other methods. During cell lysis and DNA separation from the bacterial extract, enzymes may be added to the bacterial mixture, such as proteases, for the removal of proteins, and RNases for the removal of RNA.

The vector containing the N3ICD cDNA may be purified after extraction from the cells in which it was amplified. Methods for DNA purification include ethanol precipitation, phenol-chloroform extraction, gel electrophoresis, column purification, or other methods.

In a different embodiment, the N3ICD polypeptide SEQ ID NO:2, or fragments thereof, may be generated directly in the same host cells, e.g. E. coli, used for plasmid multiplication. In this case, the N3ICD cDNA is inserted in a vector under the control of a bacterial promoter, e.g. the lac promoter, which is under the control of a transcriptional operator, e.g. the lac operator. When the lac operator is bound by the lac repressor, it prevents the binding of RNA polymerase to the lac promoter and blocks transcription. The lac repressor can be dissociated from the lac operator by the presence of allolactose or its analog beta-D-thiogalactopyranoside (IPTG), which allows the binding of RNA polymerase to the lac promoter to initiate transcription. In practice, bacteria are first allowed a period of growth, at the end of which IPTG (100 micromolar-1 milimolar) is added to the growth medium to initiate protein expression. At the end of the protein expression period, bacteria are lysed and N3ICD is extracted.

Alternatively, N3ICD may be fused with a polypeptide tag, typically at the N-terminus, in order to facilitate the extraction of N3ICD by affinity purification on appropriate columns. Examples of polypeptide tags well-known to those of ordinary skill in the art include, but are not limited to, poly-histidine (His), chitin binding protein (CBP), glutathione-S-transferase (GST), streptavidin (Strep), maltose binding protein (MBP), and others. N3ICD fused with such polypeptide tag may be purified on affinity columns appropriate for each polypeptide tag, e.g. containing nickel/cobalt ions (His tag), glutathione (GST tag), amylose agarose (MBP tag), streptavidin (Strep tag), chitin (CBP), or other columns.

When using bacterial expression systems for the production of N3ICD polypeptide, the bacteria are separated by centrifugation, filtration, or other methods, and then the bacterial pellet is lysed in a high pH medium to release the polypeptide. The bacterial debris are then separated by bringing the suspension to a neutral PH, followed by centrifugation. The N3ICD polypeptide is extracted from the soluble fraction and purified by affinity binding, on a column or in suspension, using a marker sequence that was fused to the polypeptide.

### Procedure for Generating Neurons Using N3ICD

This Invention demonstrates for the first time that Notch3 is generally associated with cellular differentiation, including but not limited to, neuronal differentiation, as opposed to the other members of the Notch receptor family. Up to the date of this invention, Notch3 has been considered to promote cellular proliferation, similarly to the other Notch receptors.

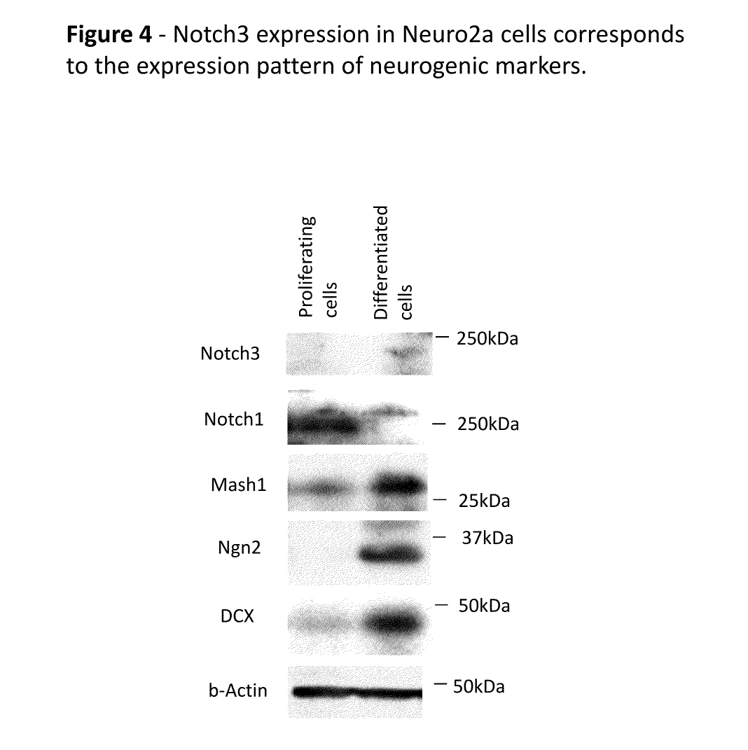

For example, the role of Notch3 in Neuro-2a cells is described. Neuro-2a cells are a mouse cancer cell line with stem cell characteristics, which proliferate when grown in a cell culture medium including Eagle's Minimum Essential Medium (EMEM) and 10% fetal bovine serum (FBS). EMEM is a cell culture medium with defined composition, described commercially and well-known to those of ordinary skill in the art. Neuro-2a cells have been used as a model system that can be induced to differentiate into neurons by eliminating FBS from their culture medium. The differentiation of Neuro-2a cells by eliminating FBS (−FBS conditions) correlates with an increase in Notch3 expression, alongside with other markers typical for neuronal differentiation, including mammalian achaete-scute homolog 1 (Mashl), neurogenin 2 (Ngn2) (Bertrand, et al., Nat. Rev. Neurosci. 3, 517-530), and doublecortin (DCX) (Francis, et al., Neuron 23, 247-256), as depicted in FIG. 4. The increase in Notch3 expression in Neuro-2a cells is opposite to the variation of other Notch family members, such as Notch 1, the expression of which is decreased in differentiated Neuro-2a cells.

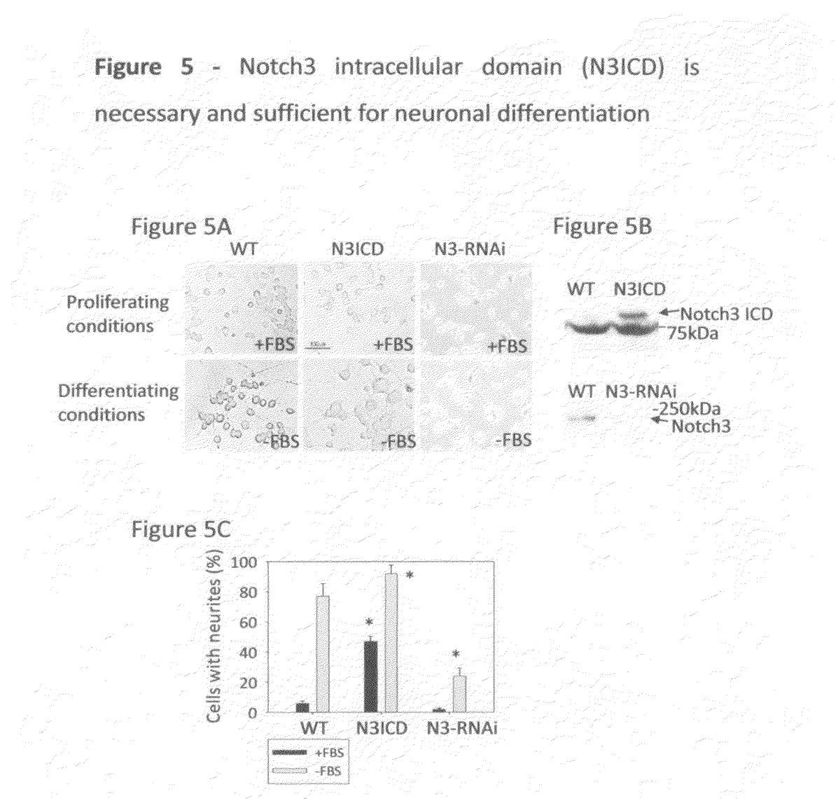

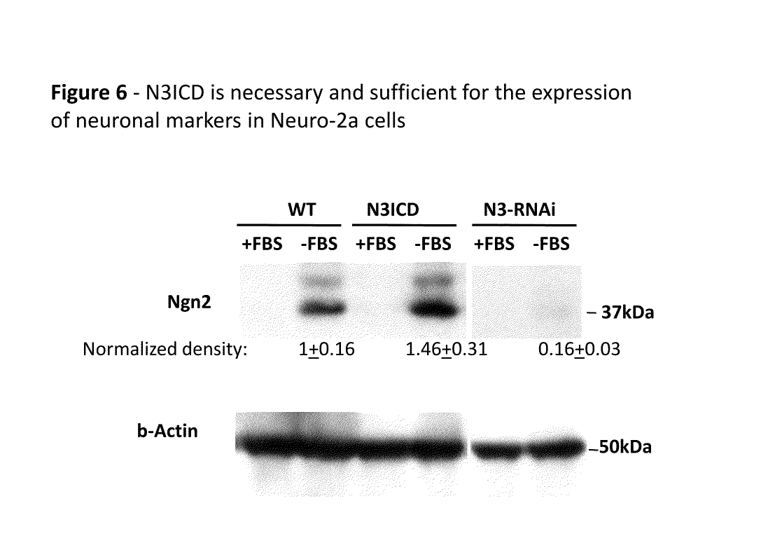

The expression of N3ICD in host cells may be naturally induced as a necessary step for the initiation and/or completion of cellular (e.g. neuronal) differentiation. For example, the prevention of the expression of N3ICD, using Notch3-specific interference RNA (N3-RNAi) inhibits the differentiation of Neuro-2a cells, as depicted in FIG. 5A-5C. In addition, the inhibition of N3ICD expression also inhibits the expression of neural marker Ngn2, as depicted in FIG. 6. In addition, in Notch3 knock-out mice, spinal cord neurons remain at an immature stage characterized by incomplete differentiation, resulting in increased pain sensitivity (Rusanescu, et al., J Cel Mol Med. 18: 2103-2114 (2014)).

The expression of N3ICD may be artificially induced in host cells for the purpose of initiating cellular differentiation into cells with a desired phenotype. The host stem cells containing N3ICD may begin to differentiate spontaneously into various cell types (e.g. neurons) within several days after the introduction of N3ICD into the cells. For example, Neuro-2a cells containing N3ICD spontaneously acquire a neuronal phenotype, represented by neurite growth even in the absence of differentiating conditions (+FBS), as depicted in FIG. 5A-5C. The neuronal phenotype acquired by Neuro-2a cells after the introduction of N3ICD is also demonstrated by an increased expression of neuronal marker Ngn2, as depicted in FIG. 6.

The vector containing the N3ICD polynucleotide sequence can be introduced into mammalian host stem cells or progenitor cells, by transfection, viral infection, electroporation, injection or other methods. The host stem cells that contain the vector with N3ICD polynucleotide may be selected from the other stem cells that do not contain the vector, using selectable markers present in the vector, which include antibiotic resistance genes (e.g. hygromycin, puromycin, geneticin, or others). The selective chemical substance corresponding to a resistance gene may be added to the cell culture medium throughout the cell multiplication process, as a selection pressure to prevent the host cells from eliminating the vector. For example, a vector containing N3ICD and a puromycin resistance gene may be introduced into Neuro-2a cells, and then the Neuro-2a cells that contain the vector may be selected by the addition of 1 microgram/ml puromycin to the cell culture medium, which would destroy the Neuro-2a cells that do not contain the vector.

Alternatively, the N3ICD polypeptide may be generated by expression from its cDNA in a cell expression system (e.g. E. coli), then isolated from the cell extract, and then introduced in the human host cells of interest using protein transfection systems, including peptide-based (Clontech, Mountain View, Calif.) or lipid-based (Life Technologies, Grand Island, N.Y.) systems.

### Host Cells

The polynucleotide sequence of the present invention, encoding N3ICD, may be expressed in any host cell appropriate for the purpose for which it is being used. Examples of host cells that can be used in the present invention include prokaryotic, yeast and eukaryotic cells. Prokaryotic cells, yeast and eukaryotic cells may be used for vector or polypeptide quantitative amplification. Human cells, other mammalian cells, and hybrid human-mammalian cells may be used for functional N3ICD protein expression, in order to treat a disease.

Examples of prokaryotic cells that can be used as host cells in the present invention include E. coli, Enterobacter, Proteus, B. subtilis, Erwinia, Klebsiella, Salmonella, Pseudomonas and Shigella. Prokaryotic expression vectors that may be used in the present invention contain one or more selectable marker genes encoding proteins that offer antibiotic resistance. Examples of vectors used in prokaryotic host cells include the pRSET (Invitrogen, Calrlsbad, Calif.) and pET (Novagen, Madison, Wis.) vectors. Promoter sequences commonly used in prokaryotic host cell expression include T7 (Rosenberg, et al., Gene 56: 125(1987)) and tac (Sambrook, et al., Molecular Cloning, A Laboratory Manual, 2^{nd} ed., Cold Spring Harbor Laboratory (1990)) promoters.

Yeasts that may be useful in the present invention include members of the genus Saccharomyces, Schizosaccharomyces, Pichia, Actinomycetes, Kluyveromycetes, Candida, Neurospora and Trichoderma. Yeast vectors typically include an origin of replication sequence, an autonomously replicating sequence, a promoter, a polyadenylation sequence, a transcription termination sequence, and a selectable marker gene. Promoter sequences commonly used in yeast include promoters for metallothionein, 3-phosphoglycerate kinase, hexokinase, enolase and others that are well known in the art (Fleer, et al., Gene 107:285 (1991)).

Mammalian host cells, including human cells, that are useful for the present invention include stem cells, progenitor cells, and generally cells that maintain the capability to proliferate or that are incompletely differentiated, including but not limited to tumor cells, experimentally-modified cell lines, and cells that are incompletely differentiated in human patients as a result of disease. Examples of experimentally-modified cell lines which are commonly used to study cellular, including neuronal differentiation include PC12, Neuro-2a, NT2, SH—SY5Y, and other cell lines. Vectors can be introduced into host cells outside or inside of an organism, using methods well known to those of ordinary skill in the art.

The host stem cells, containing a vector that includes the N3ICD polynucleotide, may be introduced into an organism (e.g. human patient) for the purpose of promoting the differentiation of these cells into neurons or other cell types.

Alternatively, the host stem cells containing the N3ICD-expressing vector may be differentiated in cell culture, for example on a cell culture dish, on a bi-dimensional or tri-dimensional scaffold, or on other artificial or natural supports, alone or in combination with other cell types, in order to create multicellular aggregates functionally similar to various types of biological tissues, including nervous tissue. Such tissues can be subsequently transplanted into human patients.

Alternatively, the host stem cells may be harvested from the intended patient (e.g. autologous) or may be collected from a different human, or from an animal donor (e.g. heterologous).

Alternatively, the vector containing the N3ICD polynucleotide may be introduced directly into the human host cells in-vivo, for example by using a viral vector, vesicles containing lipids or other chemical compounds, nanoparticles, or other types of vectors. This method may be used to promote cellular differentiation in order to treat human pathologies characterized by defective cellular differentiation. Examples of such pathologies include cancer, and neurological disorders associated with incomplete neuronal differentiation, including, but not limited to schizophrenia, bipolar disorder, epilepsy, chronic pain and other disorders.

Alternatively, N3ICD may be generated within the organism (e.g. human patient) by activation of the endogenous Notch3 receptor present in the cells of interest. The endogenous Notch3 receptor may be activated using a Notch3-specific antibody, or by artificially activating a protease enzyme, or by using a combination of one or more extracellular fragments of Notch ligands, which preferentially activate the Notch3 receptor and generate N3ICD inside the host cell.

### Vectors

Many vectors are available. A vector generally includes, but is not limited to one or more of the following components: a signal sequence, an origin of replication, an enhancer element, an inducible control element, a promoter, one or more marker genes, multiple cloning sites, and a transcription termination sequence. The expression vectors include a nucleotide sequence operably linked to appropriate transcriptional or translational regulatory nucleotide sequences such as those derived from mammalian, microbial, viral or insect genes. Examples of regulatory sequences include transcriptional promoters, operators, enhancers, RNA binding sites and/or other sequences which control transcription and translation initiation and termination. Nucleotide sequences are operably linked when the regulatory sequences are connected functionally to (e.g. control the transcription of) the nucleotide sequence coding for the polypeptide of interest (e.g. N3ICD).

In addition, sequences encoding various peptides that are not part of the natural Notch3 sequence may be incorporated into expression vectors. For example, a nucleotide sequence for a signal peptide (e.g. a nuclear localization sequence) may be fused in-frame to the N3ICD nucleotide sequence, so that it modulates the function of the N3ICD polypeptide in the host cells.

The vector may be a plasmid vector, a single or double-stranded phage vector, or a single or a double-stranded RNA or DNA viral vector. Such vectors may be introduced into cells by well-known techniques for introducing polynucleotides into cells. Phage and viral vectors may be also introduced into cells as packaged or encapsulated virus by well-known techniques of infection and transduction.

Alternatively, N3ICD may be placed under the control of an inducible promoter, which allows human control of N3ICD expression and of resulting cellular differentiation according to a desired time frame. This method allows the controlled differentiation of host stem cells in cell culture or after introduction into an organism. For example, host stem cells may be induced to differentiate immediately after being introduced in an organism, or they may be prevented to differentiate within the organism for variable periods of time, in order to allow the cells to proliferate, before being induced to differentiate. The differentiation of host stem cells may be induced within the organism by changing conditions in a manner that induces the expression of N3ICD. For example, N3ICD expression may be placed under the control of a “Tet-on” inducible system. In this example, the N3ICD promoter is fused in frame to a TetO tetracycline operator sequence that regulates N3ICD expression. TetO and N3ICD expression are activated only when TetO is bound by the tetracycline transactivator protein (tTA). tTA can bind to TetO and initiate N3ICD expression only in the presence of tetracycline or a tetracycline-related compound (e.g. doxycycline). As a result, N3ICD protein expression occurs only when doxycycline or a related compound are administered to the patient. The production of N3ICD protein will then induce the differentiation of the host cell, for example into neurons. tTA may be expressed from the same vector as N3ICD, or from different vectors, introduced simultaneously into the host cell.

Alternatively, N3ICD expression may be placed under the regulation of the dexamethasone-inducible glucocorticoid promoter, or of other types of inducible promoters.

### N3ICD Purification

The vector containing the N3ICD polynucleotide, produced in cells, can be purified from the cellular debris by centrifugation, ultrafiltration, or other techniques typically used in the art. The vector can then be separated from the supernatant by precipitation, affinity chromatography, electrophoresis, or a combination thereof. Vector precipitation and dissolution may be performed using the variations in polynucleotide solubility in different solvents, different ionic strengths or different pH values. Several purification cycles may be performed to obtain the desired purity of the vector. The purity of the vector containing the N3ICD polynucleotide can be determined using spectrometry, electrophoresis (with or without vector linearization), sequencing, or other methods commonly used in the art. During various steps of the vector separation and purification procedures, any commercially available DNase inhibitors may be added to prevent the degradation of the polynucleotide. In addition, RNase (e.g. RNase A) may be added to remove RNAs from the vector.

The N3ICD polypeptide produced in cells can be separated from the cellular debris by centrifugation, ultrafiltration, or other techniques typically used in the art. The N3ICD can be separated from the supernatant by affinity purification using a fused polypeptide tag, such as GST, His, AviTag, SBP, MBP, calmodulin, and others commonly used in the art. The tagged N3ICD polypeptide can be separated using appropriate complementary affinity molecules, bound to a solid support, such as a bead slurry or a chromatographic column. After separation, the tagged N3ICD polypeptide is washed and eluted using appropriate reagents (e.g. reduced glutathione for GST tags). Further purification techniques include dialysis, chromatography, concentration filters, lyophilization, and others. The protein tag may be removed enzymatically, e.g. using thrombin or factor Xa in the case of the GST tag. During various steps of the N3ICD polypeptide purification, protease inhibitors may be added to prevent polypeptide degradation. Protease inhibitors that may be used include phenyl-methyl-sulphonyl-chloride (PMSF), aprotinin, EDTA, or any other commercially available mixture of protease inhibitors.

### Pharmaceutical Formulation

Therapeutic formulations of the N3ICD polynucleotide or polypeptide may be prepared for storage as lyophilized formulations or aqueous solutions, by mixing the purified polynucleotide or polypeptide with optional carriers, excipients or stabilizers commonly used in the art, all of which are termed “excipients”. Excipients include buffers, stabilizing agents, anti-oxidants, preservatives, detergents, salts, and other additives. Such additives must be nontoxic to cells or recipients at the dosages and concentrations used.

Buffering agents maintain the pH of the N3ICD formulation in a range which approximates physiological conditions. Suitable buffering agents for use with the current invention include organic and/or inorganic acids and salts thereof, such as citrate buffers (e.g. monosodium citrate-disodium citrate mixture, citric acid-trisodium citrate mixture, etc.), succinate buffers (e.g. succinic acid-monosodium succinate mixture, succinic acid-sodium hydroxide mixture, etc.), fumarate buffers (e.g. fumaric acid-sodium hydroxide mixture, fumaric acid-disodium fumarate mixture, etc.), gluconate buffers (e.g. gluconic acid-sodium gluconate mixture, gluconic acid-sodium hydroxide mixture, etc.), acetate buffers (e.g. acetic acid-sodium acetate mixture, acetic acid-sodium hydroxide mixture, etc.), phosphate buffers (e.g. monosodium phosphate-disodium phosphate mixture, etc.), trimethylamine salts (e.g. Tris), and other buffers.

Preservatives may be used to inhibit microbial growth in the formulation. Suitable preservatives for use with the current invention include phenol, benzyl alcohol, meta-cresol, methyl paraben, propyl paraben, benzalkonium halides, catechol, resorcinol, cyclohexanol, and others typically used in the art.

Stabilizers may be used to increase solubility, provide isotonicity, prevent denaturation, or prevent adherence to container of the N3ICD polypeptide or N3ICD polynucleotide. Suitable stabilizers for use with the current invention include polyhydric alcohols and sugars (e.g. glycerin, polyethylene-glycol, erythritol, xylitol, mannitol, sorbitol, inositol, trehalose, lactose, etc.), amino-acids (e.g. arginine, glycine, histidine, polypeptides, etc.), proteins (e.g. albumin, gelatin, etc), reducing agents (e.g. urea, glutathione, thioglycerol, sodium thioglycolate, sodium thiosulfate, etc.), and others commonly used in the art.

Detergents may be used to increase solubility and prevent aggregation of the formulation. Suitable detergents for use with the current invention include polysorbates (e.g. 20, 80, etc.), polyoxyethylene sorbitan ethers (TWEEN-20, TWEEN-80), polyoxamers and others commonly used in the art.

The formulations for in-vivo use must be sterile. This can be achieved by filtration through sterile filtration membranes.

### Articles of Manufacture

In another embodiment of the invention, an article of manufacture is provided, containing materials useful for the treatment of the disorders described in the invention. The article of manufacture comprises a label and a container. Suitable containers include vials, bottles, syringes, and test tubes. The containers may be formed from a variety of materials, such as glass or plastic. The container holds a composition which is effective in treating a disorder or in modifying cells used to treat a disorder. The active component in the composition is N3ICD, in the form of a vector or a polypeptide. The label attached to the container indicates that the composition is used to treat the condition of choice. The article of manufacture may further include a second container comprising a pharmaceutically acceptable buffer, such as phosphate-buffered saline, dextrose solution or Ringer's solution. The article of manufacture may further include a third container comprising a pharmaceutically acceptable cell transfection system (e.g. liposomes, etc.). The article of manufacture may further include other materials necessary for the user, including other buffers, antibiotics, filters, syringes, and instructions for use.

### Therapeutic Uses of N3ICD

It is intended that N3ICD described in the current invention may be used to treat a mammal. In one embodiment, N3ICD polynucleotide may be administered to a mammal to treat a disorder or disease. The present invention is directed to generate neurons or other cell types in order to replace cells lost to injury or disease. Proliferating host cells, including stem cells, progenitor cells, cancer cells, may be extracted from the same individual (autologous), or from another individual of the same species, or from a different species (heterologous). N3ICD may be introduced in these host cells, or in artificially modified cell lines, in the form of a polynucleotide that has the ability to generate the N3ICD polypeptide by transcription in-vivo, inside the host cell. The host cells that express the N3ICD polypeptide may be introduced back into the same or into a different host mammal, by transplantation at, or near the site affected by injury or disease, where the host cells are intended to differentiate as a result of N3ICD function.

In cases where host cells or tissues expressing N3ICD are transplanted into a receiving individual different from the original individual donor of the host cells, the receiving individual may be administered immune suppression therapy in order to avoid the rejection of transplanted cells or tissues.

In a different embodiment, the N3ICD nucleic acid sequence may be administered in the form of a viral vector, or in other forms of gene therapy, to proliferating cells in a mammal for the purpose of becoming intracellular, expressing N3ICD polypeptide and inhibiting cell proliferation by inducing cellular differentiation. For example, vectors expressing N3ICD polynucleotide may be introduced in a tumor in order to inhibit the progression of cancer. Viral vectors that may be used for N3ICD polynucleotide delivery to cells inside a mammal include adenoviruses, adeno-associated viruses, retroviruses, and other types of viruses. Transfecting agents, encapsulation in liposomes, microparticles, microcapsules, or administration in linkage to a ligand subject to receptor-mediated endocytosis may be also used to introduce N3ICD nucleic acid sequence into cells, inside or outside a mammal. Alternatively, nucleic acid-ligand complexes can be formed, in which the ligand comprises a fusogenic peptide to disrupt endosomes, allowing the nucleic acid to avoid lysosomal degradation. Alternatively, the nucleic acid may be targeted for in vivo cell specific uptake and expression, by targeting a specific receptor.

In another embodiment, the N3ICD polypeptide may be introduced into proliferating host cells, for the purpose of regenerating lost cells, e.g. neurons, or to inhibit cell proliferation. The N3ICD polypeptide may be introduced into host cells outside of a mammal, followed by the transplantation of the N3ICD-modified cells into the mammal, or may be introduced directly into the host cells inside the mammal.

In a different embodiment, the host cells may be stimulated to produce their own Notch3 receptor and/or cleave their own Notch3 receptor to produce N3ICD, for the purpose of inducing cellular differentiation. This procedure may be done using inducers of transcription specific for the Notch3 receptor, or by using antibodies that specifically bind to the extracellular domain of Notch3 and activate Notch3 cleavage (Li, et al., J. Biol. Chem. 283, 8046-54 (2008)), or by inducing Notch3-specific proteases of the ADAM metallopeptidase or presenillin enzyme families (Artavanis-Tsakonas S, et al., Science 284, 770-776 (1999)), or by using a combination of one or more Notch3-specific extracellular binding domains of Notch ligands. This activation of cellular Notch3 receptors may be performed on cells in cell culture or directly in cells inside a mammal.

Alternatively, cells induced to express N3ICD may be first differentiated in cell culture, alone or in combination with other cell types, on a bi-dimensional or tri-dimensional scaffold, in order to assemble into functional tissues, which may be subsequently transplanted into a mammal.

In a different embodiment, cells induced to express N3ICD, whether by introducing a vector containing N3ICD polynucleotide, or by directly introducing the N3ICD polypeptide, may be introduced in an experimental animal for pre-clinical studies designed to develop a treatment for a disease or disorder. Alternatively, N3ICD polynucleotide or N3ICD polypeptide may be introduced directly into an experimental animal with the intention of being introduced into cells within the animal, using any methods well known to those of ordinary skill in the art.

### EXAMPLES

### Example 1

### Generation of Notch3 Intracellular Domain (N3ICD) cDNA

Notch3 cDNA sequence was analyzed using an on-line public database (National Center for Biotechnology Information). Human total liver RNA (Life Technologies, Grand Island, N.Y.) was used as template to synthesize the first strand of cDNA using a commercially available cDNA synthesis kit. Notch3-specific primers to regions outside the Notch3 intracellular domain were used to amplify cDNA from Notch3 mRNA. In a second step, primers complementary to the N-terminal and C-terminal ends of N3ICD, extended with linkers including in-frame, non-identical restriction sites corresponding to two different restriction enzymes (e.g. HindIII and ClaI), were used to amplify N3ICD by PCR. The PCR reaction product was treated with HindIII and ClaI, then separated on an agarose gel. The identity of the cDNA obtained was verified against a standard oligonucleotide ladder mixture of known molecular sizes.

### Example 2

### Generation of a Plasmid Vector, Containing the N3ICD Polynucleotide Sequence

The purified N3ICD cDNA was ligated in-frame, using a ligase enzyme, in a plasmid which also comprises an ampicillin and a puromycin resistance elements (e.g. pBABEpuro), and which was also previously treated with the same pair of restriction enzymes. Typically, the two restriction enzymes used are selected in manner that generates non-matching ends on the vector, in order to prevent the vector from ligating back on itself in the absence of the insert containing the polynucleotide of interest. After ligation, the pBABEpuro plasmid containing the N3ICD insert was introduced by transformation into E. coli, and then the E. coli bacteria were spread on an agar plate containing ampicillin. One of the surviving E. coli colonies, which is expected to contain the vector fused with the N3ICD insert, is selected and further grown in LB broth containing ampicillin. The LB broth is a standard bacterial culture medium, containing tryptone, yeast extract, sodium chloride and other components, which may be added in varying compositions.

After E. coli have multiplied in culture to reach a specific optical density of the medium, the bacteria are separated by centrifugation, and the plasmid was extracted using a commercially available DNA extraction kit. After purification, the polynucleotide sequence within the plasmid corresponding to N3ICD was checked for possible errors by DNA sequencing, using primers corresponding to the two ends of the polynucleotide sequence, and compared against published Notch3 nucleotide sequence, using publicly available computer software (e.g. Blast).

### Example 3

### Transfection of Neuro-2a Cells with a Plasmid Expressing N3ICD

The Neuro-2a mouse neuroblastoma cell line is an example of proliferating cells that can be induced to differentiate by expressing the N3ICD polypeptide. The transfection of Neuro-2a with a vector containing the N3ICD polynucleotide can be performed through any available method well known to those of ordinary skill in the art. In this particular case, a pBABEpuro plasmid containing the N3ICD cDNA insert was transfected into Neuro-2a cells using Lipofectamine 2000, a commercially available liposome transfection kit (Life Technologies, Grand Island, N.Y.). After transfection, the cells that have incorporated the vector are selected by adding puromycin to the cell culture medium. Cell colonies are selected and grown individually, then each cell colony is tested by Western blot for the expression of the N3ICD polypeptide, in comparison with the original Neuro-2a cells, as depicted in FIG. 5B.

### Example 4

### Induced Differentiation of Neuro-2a Cells that Express the N3ICD Polypeptide

Wild-type (unmodified) Neuro-2a cells are typically grown in culture in a medium containing EMEM and 10% FBS. The Neuro-2a cells can be induced to differentiate into neurons by removing the FBS-containing medium and replacing it with EMEM alone (−FBS), as depicted in FIG. 5A. This differentiation process under (−FBS) conditions is associated with a decrease in Notch1 protein level, as well as increases in protein levels for Notch3 and other proteins associated with neuronal differentiation, including Mashl, Ngn2 and DCX, as depicted in FIG. 4. The transfection and expression of N3ICD in Neuro-2a cells induces these cells to differentiate even under non-differentiating conditions, as depicted in FIG. 5A-5C. This supports the role of N3ICD as an inducer of cellular (e.g. neuronal) differentiation. In addition, although the N3ICD transfected in this example was of human origin, the expressed N3ICD was capable of performing its intended action of inducing the differentiation of Neuro-2a cells of mouse origin. According to this experiment, the N3ICD polypeptide corresponding to different species maintains its physiological action across species, despite some differences in the polypeptide sequence, as depicted in FIG. 3A-B.

Alternatively, Neuro-2a cells or stem cells transfected with a vector that expresses N3ICD may be introduced in a mammal for the purpose of differentiating into various cell types within that mammal. The type of cell produced upon differentiation by N3ICD expression may depend on both the type of proliferating cell (e.g. tumor cell, embryonic stem cell, mesenchymal stem cell, iPSC) as well as on the type of tissue that the host cell is transplanted into. For example, adult neural progenitor cells obtained from adult mouse spinal cord retain multipotency, the ability to differentiate into multiple cell types in culture, including neurons, astrocytes and oligodendrocytes. However, when these progenitor cells were transplanted into mouse spinal cord, they differentiated exclusively into glial cells (Shiahbuddin, et al., J. Neurosci. 20, 8727-35 (2000)). Because Notch3 is expressed specifically in adult spinal cord neurons, but not in glial cells (Rusanescu, et al., J Cel Mol Med. 18, 2103-2114 (2014)), it is expected that the expression of N3ICD in stem cells transplanted into a mammal would redirect the differentiation of these stem cells preferentially into neurons.

### Example 5

### Generation of a Vector Expressing N3ICD Under the Control of the Tet-on Promoter

The expression of N3ICD may be optionally induced in a controlled manner by placing the N3ICD polynucleotide in an inducible vector, e.g. the commercially available Tet-on system (Clontech, Mountain View, Calif.). For this purpose, the N3ICD cDNA, obtained as in Example 1, was introduced in a Tet-on vector following a similar procedure as described in Example 2. After amplification, the integrity of the N3ICD polynucleotide sequence was tested by DNA sequencing, using appropriate markers. Alternative commercially available Tet-on plasmids may be used that express a fluorescent marker, e.g. pTRE3G-ZsGreen or pTRE3G-mCherry (Clontech, Mountain View, Calif.), for identification in cell culture or post-mortem in the mammal in which it was introduced. Instead of a Tet-on promoter, other inducible promoters may be used, including Tet-off, glucocorticoid, or other promoters, which may be induced by the appropriate chemical compounds.

### Example 6

### Transfection of Neuro-2a Cells with a N3ICD-Containing Tet-on Plasmid

A stable Neuro-2a cell line that expresses N3ICD only upon controlled induction, was generated by co-transfecting Neuro-2a cells with the Tet-on plasmid containing the N3ICD polynucleotide obtained in example 5, and an antibiotic selection marker, pBABEpuro, in a vector-to-marker molar ratio of 20:1. Neuro-2a cells that express the selection marker were selected by adding puromycin to the culture medium. The surviving colonies were individually tested for the expression of N3ICD by adding doxycycline to a test sample of each colony, and verifying N3ICD expression by Western blot.

### Example 7

### Differentiation of Neuro-2a/Tet-on/N3ICD Cells

Neuro-2a cells that express N3ICD under the control of a Tet-on promoter were induced to differentiate in culture by the addition of doxycycline to the cell culture medium. The expression of N3ICD in the differentiated Neuro-2a cells was identified by Western blot three days after the addition of doxycycline, demonstrating the role of N3ICD in neuronal differentiation. Instead of doxycycline, other tetracycline-related compounds may be used.

Neuro-2a cells or stem cells that express N3ICD under the control of an inducible promoter can be transplanted into a mammal, for example by direct injection into the mouse spinal cord. Various mouse strains may be used, for example nude mice that have a reduced immune reaction to the transplantation of foreign cells, which eliminates the need to administer immunosuppressive therapy to prevent transplant rejection. The mice receiving the transplant may be treated with doxycycline injections immediately, or sometime after the transplant, in order to allow transplanted cells to multiply before undergoing differentiation. A vector containing a fluorescent marker (e.g. mCherry, ZsGreen) may be used for N3ICD expression, in order to analyze post-mortem the types of cells produced by Neuro-2a differentiation. The phenotypes and proportion of the differentiated cells produced by the transplanted cells may be determined by analyzing the overlap of the fluorescent marker expressed by the transplanted cells with markers specific for each cell phenotype (e.g. neurons, astrocytes, oligodendrocytes).

### Example 8

### Expression of N3ICD in a Viral Vector

The increased expression of N3ICD in cells and tissues within a mammal can be achieved by gene therapy, for example by introducing N3ICD in a viral vector, which allows a direct insertion of N3ICD cDNA into the target cells. This procedure may be used to induce the differentiation of tumor cells, or of other cells that are incompletely differentiated within the mammal, thereby causing a disease or disorder of the mammal. In this example a recombinant adeno-associated virus (rAAV) vector is described, but other viral vectors may be used, including, but not limited to adenovirus, retroviruses such as lentivirus, or other viruses. The rAAV method described in the invention involves the introduction of the N3ICD polynucleotide sequence in a rAAV vector carrying the inverted terminal repeats of the AAV genome and the green fluorescent protein (GFP) gene. The rAAV vector containing N3ICD is then co-transfected with a second plasmid that carries the Rep-Cap genes, and with a third plasmid which encodes the AAV helper genes, into host cells for AAV production, e.g. HEK293 cells. The cell culture medium containing the AAV particles which encode N3ICD is collected, and the AAV is purified by gradient centrifugation and chromatographic column purification. The AAV vector expressing N3ICD and the GFP marker was injected into a mammal, e.g. in mouse spinal cord. The mice were sacrificed one week later by perfusion-fixation. Spinal cord sections at the injection site were analyzed by fluorescence microscopy for GFP expression.

### Example 9

### Generation of N3ICD Polypeptide

The N3ICD polynucleotide was inserted, using molecular biology techniques commonly known to those of ordinary skill in the art, in a commercially available pGEX2Tk plasmid (GE Healthcare, Marlborough, Mass.), which also includes the glutathione-S-transferase (GST) protein tag. Many other lac operon-containing plasmids may be used, which express different antibiotic resistance genes and/or different protein tags for protein separation. The pGEX plasmid containing the N3ICD polynucleotide and GST tag was introduced in E. coli strand BL21 by transformation and the bacteria were spread on an agar plate containing ampicillin. The BL21 strain has the advantage of being protease deficient, however any appropriate bacterial strain commonly used in molecular biology may be used. A bacterial colony containing the plasmid was selected from the agar plate and amplified in LB broth, then the bacterial culture was treated with IPTG to induce protein expression. The bacteria were separated by centrifugation and lysed according to one of commonly used standard protocols (e.g. lysozyme, sonication, sodium hydroxide, or other methods). The supernatant was separated by centrifugation and mixed gently with a slurry of glutathione sepharose 4B beads (Dharmacon, Lafayette, Colo.). The sepharose beads were separated from the supernatant, then washed with phosphate buffer saline. The GST-N3ICD polypeptide was eluted from the sepharose beads with a glutathione elution buffer. The fused GST-N3ICD polypeptide concentration was quantified by Coomassie staining, relative to protein standards of known concentration. Throughout this process protease and phosphatase inhibitors may be added to the bacterial lysate. This example is not limiting, and is understood to include a large number of protocol variations, including different inducible plasmid systems, different protein tags, different cellular strains (e.g. bacteria, insect, or mammal), different cell lysis methods, and different protein separation methods.

### Example 10

### Introduction of N3ICD Polypeptide in Cells

The N3ICD polypeptide may be introduced in cells with the purpose of inducing cellular differentiation. The polypeptide tag used for N3ICD separation and purification (e.g. GST, His, or others) may be removed prior to introduction in a cell, by enzymatic cleavage at the tag fusion site (e.g. thrombin in the case of the pGEX2Tk plasmid). After removal of the GST tag by thrombin cleavage and chromatographic separation, the N3ICD polypeptide was introduced into Neuro-2a cells in culture using the Xfect Protein Transfection Reagent (Clontech, Mountain View, Calif.). Alternatively, other protein transfection systems may be used. Within a few days, the Neuro-2a cells developed neurites similar to the cells transfected with the N3ICD polynucleotide. It is understood that the N3ICD polypeptide transfection of other proliferating cells, including stem cells, will similarly induce their differentiation. N3ICD may also be introduced in cells which will be subsequently transplanted into a mammal for the purpose of generating differentiated cells, e.g. neurons.

### REFERENCES

Abematsu M., et al. (2010) Neurons derived from transplanted neural stem cells restore disrupted neuronal circuitry in a mouse model of spinal cord injury. J Clin Invest. 120, 3255-66.
Artavanis-Tsakonas S, et al. (1999). Notch signaling: cell fate control and signal integration in development. Science 284, 770-776.
Barnabe-Heider F., et al. (2010). Origin of new glial cells in intact and injured adult spinal cord. Cell Stem Cells 7, 470-482.
Beatus P, et al. (1999). The Notch3 intracellular domain represses Notch 1-mediated activation through Hairy/Enhancer of split (HES) promoters. Development 126, 3925-3935.
Bellavia D., et al. (2000). Constitutive activation of NF-kappaB and T-cell leukemia/lymphoma in Notch3 transgenic mice. EMBOJ. 19, 3337-48.
Bertrand N., et al. (2002). Proneural genes and the specification of neural cell types. Nat Rev Neurosci. 3, 517-530.
Boulis N, et al. (2012) Translational stem cell therapy for amyotrophic lateral sclerosis. Nature Reviews Neurology 8, 172-176.
Chi L., et al. (2006). Motor neuron degeneration promotes neural progenitor cell proliferation, migration, and neurogenesis in the spinal cords of amyotrophic lateral sclerosis mice. Stem Cells 24, 34-43.
Dang L., et al. (2006). Notch3 signaling promotes radial glial/progenitor character in the mammalian telencephalon. Dev Neurosci. 28, 58-69.
Doetsch F, Caine I, Lim D A, et al. (1999). Subventricular zone astrocytes are neural stem cells in the adult mammalian brain. Cell 97, 703-16.
Freed C R, et al. (1990). Therapeutic effects of human fetal dopamine cells transplanted in a patient with Parkinson's disease. Prog Brain Res. 82, 715-21.
Fleer R., et al., (1991). High-level secretion of correctly processed recombinant human interleukin-1 beta in Kluyveromyces lactis. Gene 107, 285-95.
Francis F, et al. (1999). Doublecortin is a developmentally regulated, microtubule-associated protein expressed in migrating and differentiating neurons. Neuron 23, 247-256.
Glass J. D., et al. (2012) Lumbar intraspinal injection of neural stem cells in patients with amyotrophic lateral sclerosis: results of a phase I trial in 12 patients. Stem Cells 30, 1144-51.
Hefferan M P, et al. (2012). Human neural stem cell replacement therapy for amyotrophic lateral sclerosis by spinal transplantation. PLOS One 7, e42614.
Horner P. J., et al. (2000). Proliferation and differentiation of progenitor cells throughout the intact adult rat spinal cord. J. Neurosci. 20, 2218-28.
Hugnot J. P., Franzen R. (2011). The spinal cord ependymal region: a stem cell niche in the caudal central nervous system. Frontiers Biosci. 16, 1044-59.
Jan Y. N., and Jan L. Y. (1994). Genetic control of cell fate specification in Drosophila peripheral nervous system. Annu Rev Genet. 28, 373-393.
Kim J H, et al. (2002). Dopamine neurons derived from embryonic stem cells function in an animal model of Parkinson's disease. Nature 418, 50-6.
Li, K., et al. (2008). Modulation of Notch signaling by antibodies specific for the extracellular negative regulatory region of NOTCH3. J Biol Chem. 283, 8046-54.
Lunn J S, et al. (2011). Stem cell technology for neurodegenerative diseases. Ann Neurol. 70, 353-61.
Nori S., et al. (2011) Grafted human-induced pluripotent stem-cell-derived neurospheres promote motor functional recovery after spinal cord injury in mice. PNAS 108, 16825-30.
Papadeas S. T., Maragakis N. J. (2009). Advances in stem cell research for amyotrophic lateral sclerosis. Curr Opin Biotechnol. 20, 545-51.
Pierfelice T. J., et al. (2011). Notch3 activation promotes invasive glioma formation in a tissue site-specific manner. Cancer Res. 71, 1115-25.
Rahman M T, et al. (2012). Notch3 overexpression as potential therapeutic target in advanced stage chemoresistant ovarian cancer. Am J Clin Pathol. 138, 535-44.
Rosenberg A. H., et al. (1987). Vectors for selective expression of cloned DNAs by T7 RNA polymerase. Gene 56, 125-35.
Rusanescu G., Mao, J. (2014). Notch3 is necessary for neuronal differentiation and maturation in the adult spinal cord. J. Cell. Mol. Med. 18, 2103-2116.
Sambrook J, et al. (1990). Molecular Cloning: a laboratory manual. 2^{nd} ed., Cold Spring Harbor Laboratory.
Shihabuddin L. S., et al. (2000). Adult spinal cord stem cells generate neurons after transplantation in the adult dentate gyrus. J. Neurosci. 20, 8727-35.
Shimizu K, et al. (2002). Functional diversity among Notch1, Notch2, and Notch3 receptors. Biochem Biophys Res Commun. 291, 775-9.
Skalova S, et al. (2015). Induced pluripotent stem cells and their use in cardiac and neural regenerative medicine. Int J Mol Sci. 16, 4043-67.
Suzuki M., Svendsen C. N. (2008). Combining growth factor and stem cell therapy for amyotrophic lateral sclerosis. Trends Neurosci. 31, 192-8.
Tanigaki K, Nogaki F, Takahashi J, et al. (2001). Notch1 and Notch3 instructively restrict bFGF-responsive multipotent neural progenitor cells to an astroglial fate. Neuron 29, 45-55.
Teng Y D, et al. (2012). Multimodal actions of neural stem cells in a mouse model of ALS: a meta-analysis. Sci Transl Med. 4, 165ra164.
Tsuji O., et al. (2010) Therapeutic potential of appropriately evaluated safe-induced pluripotent stem cells for spinal cord injury. PNAS 107, 12704-9.
van Es J H, Clevers H. (2005). Notch and Wnt inhibitors as potential new drugs for intestinal neoplastic disease. Trends Mol Med. 11, 496-502.

1. I claim a method of inducing differentiation in a cell comprising the step of inducing the expression of the intracellular domain of Notch3 receptor SEQ ID NO:2 in that cell from an exogenous vector containing the polynucleotide sequence corresponding to the Notch3 intracellular domain SEQ ID NO:1.

2. The method of claim 1, wherein the differentiated cell is a neuron.

3. The method of claim 1, wherein the differentiated cell is a bone cell.

4. The method of claim 1, wherein the purpose of inducing cellular differentiation is to inhibit cellular proliferation.

5. The method of claim 1, wherein the expression of the intracellular domain of Notch3 in cells is controlled by an inducible promoter.

6. The method of claim 1, wherein the polynucleotide sequence corresponding to the intracellular domain of Notch3 SEQ ID NO:1 is replaced by a nucleotide sequence comprising the expression of a subdomain or combination of subdomains of Notch3 intracellular domain, which induces cellular differentiation in a cell.

7. The method of claim 1, wherein the polynucleotide sequence corresponding to the intracellular domain of human Notch3 SEQ ID NO:1 is replaced by a nucleotide sequence corresponding to the intracellular domain of Notch3 from an animal, or subdomains thereof, which induces cellular differentiation after expression in a cell.

8. The method of claim 1, wherein the expression of the intracellular domain of Notch3 receptor comprises the step of activating endogenous Notch3 cleavage, using Notch3-specific transcriptional activators, antibodies, proteases or soluble fragments of Notch ligands or synthetic mimetics thereof.

9. The method of claim 1, wherein the cell expressing the intracellular domain of Notch3 receptor is introduced in a mammal for the purpose of replacing damaged cells, treating a disorder or a disease.

10. The method of claim 1, wherein the vector is a virus used to express the Notch3 intracellular domain in cells inside or outside a mammal.

11. The method of claim 1, wherein the cells expressing the intracellular domain of Notch3 are used to generate multicellular aggregates or tissues for the purpose of transplantation in a mammal.

12. The method of claim 1, wherein the cells expressing the intracellular domain of Notch3 are used in combination with electronic circuits.

13. The method of claim 1, wherein the intracellular domain of Notch3, or subdomains thereof, are introduced in cells in the form of polypeptides SEQ ID NO:2.
